(19) United States
(12) Patent Application Publication
(10) Pub. No.: US 2010/0140465 A1
(43) Pub. Date: Jun. 10, 2010

(54) Apparatus and Method for Filtration to Enhance the Detection of Peaks

(76) Inventors: Chulso Moon, Lutherville,, MD (US); Atsushi Takano, Tokyo (JP)

Correspondence Address: HOWREY LLP - East, C/O IP DOCKETING DEPARTMENT, 2941 FAIRVIEW PARK DR, SUITE 200, FALLS CHURCH, VA, 22042-2924, US

(21) Appl. No.: 12/225,704

(22) PCT Filed: Mar. 29, 2007
(86) PCT No.: PCT/US07/07855

§ 371 Date: Jan. 29, 2010

Related U.S. Application Data
(63) Continuation-in-part of application No. 11/391,183, filed on Mar. 29, 2006, now abandoned.

(51) Int. Cl. A61B 5/00; H01J 49/26

(52) U.S. Cl. 250/282

(57) ABSTRACT

Filters and methods for enhancing the identification of peaks in mass spectroscopy data are disclosed. In particular, the invention encompasses methods using hole array filters for the purpose of purifying biological fluids to be used in generating mass spectra data. The methods of the present invention may be used for enhancing relevant peaks in mass spectra data for use in identifying and diagnosing diseases or for predicting responses to particular disease treatments.

### FIELD OF THE INVENTION

The present invention relates to methods of enhancing the identification of peaks in mass spectra data for use in the early prediction, detection, and response to treatment of diseases in a human.

### BACKGROUND OF THE INVENTION

The health of a cell and of an organism is reflected by the proteins and other molecules that it contains. The detection, identification, and quantification of proteins and other molecules, such as lipids and carbohydrates, may facilitate disease mechanism elucidation, early detection of disease, prediction of disease, and evaluation of treatments.

Recent advances in genomics research have led to the identification of numerous genes associated with various diseases. However, while genomics research can identify genes associated with a genetic predisposition to disease, there is still a need to characterize and identify markers such as proteins that may be present in an individual patient. A “marker” typically refers to a polypeptide or some other molecule that differentiates one biological status from another. Recently developed methods for molecule detection have made it possible to measure a large fraction of these molecules, opening up a range of new, targeted methods for disease detection, prevention, and treatment. To effectively practice such methods requires the ability to identify individual molecules or markers, often at low concentrations, from mixtures of hundreds or thousands of different compounds.

The use of mass spectrometric methods is replacing gels as the method of choice for bioassays. Exemplary mass spectrometric formats include matrix assisted laser desorption/ionization mass spectrometry (MALDI), see, e.g., U.S. Pat. No. 5,118,937 and U.S. Pat. No. 5,045,694, and surface enhanced laser desorption/ionization mass spectrometry (SELDI), see, e.g., U.S. Pat. No. 5,719,060. The great advantage of mass spectrometry over other technologies for global detection and monitoring of subtle changes in cell function is the ability to measure rapidly and inexpensively thousands of elements in a few microliters of biological fluid. For example, disease processes that result from altered genes, such as cancer, produce altered protein products that circulate in the blood as polypeptides and other molecules of varying size. Mass spectrometry allows for the detection of such products and the subsequent diagnosis and analysis of the disease.

Although many mass spectrometric patterns of complex fluids such as serum defy visual analysis, computational approaches have been used to distinguish subtle differences in patterns from affected individuals compared with unaffected individuals. Efforts to improve the sensitivity of assays have resulted in the application of a number of mass spectrometric formats to the analysis of samples of biological relevance. In addition to the innovations in mass spectrometric techniques, substrates that adsorb an analyte (“chips”) have also been developed and the early designs have been improved upon. However, these methods have thus far proven insufficient to improve the sensitivity of mass spectrometric assays to acceptable levels.

Thus, there exists a need for methods of improving the sensitivity of mass spectrometric assays as they are used in methods of early disease diagnosis, disease prediction, monitoring disease progression or response to treatment, and in identifying which patients are most likely to benefit from particular treatments.

### SUMMARY OF THE INVENTION

The present invention relates to methods of enhancing the identification of peaks in mass spectra data for use in the early prediction, detection, and response to treatment of diseases in a human.

One embodiment of the present invention includes a method for determining the probability of disease. The method may comprise the steps of filtering a biological fluid through a hole array filter, generating mass spectra data from the filtered biological fluid, and comparing the mass spectra data with a database.

Yet another embodiment of the present invention includes a method of predicting response to disease treatment. The method may comprise the steps of generating a first set of mass spectra data from a first set of samples from a population that responds to a treatment of a disease A after filtration of the first set of samples through a hole array filter and generating a second set of mass spectra data from a second set of samples from a population that does not respond to the same treatment of disease A after filtration of the second set of samples through a hole array filter. The method may also include the step of comparing corresponding peaks in first and second sets of mass spectra data, wherein a difference in corresponding peaks indicates that the peaks represent at least one marker indicating the likelihood of response to the treatment of disease A. Upon identifying the at least one marker, the at least one marker may be used to predict the likelihood of response to the treatment of disease. In one embodiment, the structure of the hole array filter through which the first set of samples are filtered and the structure of the hole array filter through which the second set of samples are filtered are substantially identical. In another embodiment, the hole array filter through which the first set of samples are filtered and the hole array filter through which the second set of samples are filtered are separate hole array filters. In another embodiment, each samples is filtered through a separate hole array filter. Another embodiment of the present invention includes a method of enhancing the identification of peaks in a mass spectrometric method. The method may comprise the steps of filtering a sample through a hole array filter and generating mass spectra data from the sample.

Yet another embodiment of the present invention may include a method of increasing sensitivity and specificity in disease detection. The method may comprise the steps of generating a first set of mass spectra data from a first set of biological fluid samples from a population with disease A after filtration of the first set of biological fluid samples through a hole array filter and generating a second set of mass spectra data from a second set of biological fluid samples from a population without disease A after filtration of the second set of biological fluid samples through a hole array filter. The method may also include the step of comparing the first and second sets of mass spectra data, wherein a difference between corresponding peaks in the first and second sets of mass spectra data indicates at least one disease A negative marker. In one embodiment, the structure of the hole array filter through which the first set of samples are filtered and the structure of the hole array filter through which the second set of samples are filtered are substantially identical. In another embodiment, the hole array filter through which the first set of samples are filtered and the hole array filter through which the second set of samples are filtered are separate hole array filters. In another embodiment, each samples is filtered through a separate hole array filter.

Further, one embodiment of the present invention may include an apparatus for filtering biological fluid to predict response to disease treatment comprising at least one hole array filter. A first set of samples from a population that respond to a treatment of a disease A may be filtered through the at least one hole array filter and a first set of mass spectra data may be generated from the first set of samples after filtering through the at least one hole array filter. A second set of samples from a population that does not respond to the same treatment of disease A may also be filtered through the at least one hole array filter and a second set of mass spectra data may be generated from the second set of samples after filtering through the at least one hole array filter. Additionally, corresponding peaks in the first and second sets of mass spectra data may be compared, wherein a difference in corresponding peaks may indicate that the peaks represent at least one marker indicating the likelihood of response to the treatment of disease A. In one embodiment, the at least one hole array filter includes at least one first hole array filter and at least one second hole array filter, each having substantially identical structure. The at least one first hole array filter may be used for filtering the first set of samples and the at least one second hole array filter may be used for filtering the second set of samples. Each sample may be filtered through a separate hole array filter.

Further, one embodiment of the present invention may include an apparatus for filtering biological fluid to detect disease by measuring mass spectra data of filtered biological fluid comprising at least one hole array filter. A first set of biological fluid samples from a population with disease A may be filtered through the. at least one hole array filter and a first set of mass spectra data may be generated from the first set of biological fluid samples after filtering through the at least one hole array filter. A second set of biological fluid samples from a population without disease A may also be filtered through the at least one hole array filter and a second set of mass spectra data may be generated from the second set of biological fluid samples after filtering through the at least one hole array filter. Additionally, the first and second sets of mass spectra data may be compared, wherein a difference between corresponding peaks in the first and second sets of mass spectra data may indicate at least one disease A negative marker. In one embodiment, the at least one hole array filter includes at least one first hole array filter and at least one second-hole array filter, each having substantially identical structure. The at least one first hole array filter may be used for filtering the first set of samples and the at least one second hole array filter may be used for filtering the second set of samples. Each sample may be filtered through a separate hole array filter.

Further, one embodiment of the present invention may include an apparatus for filtering biological fluid to enhance the identification of peaks in a mass spectrometric method comprising a hole array filter. A biological fluid sample may be filtered through the hole array filter, and mass spectra data may be generated from the filtered biological fluid sample.

Further, one embodiment of the present invention may include a method for detecting at least one negative marker for detecting a disease. The method may comprise steps of generating a first set of mass spectra data from a first set of biological fluid samples from a population with the disease after filtration of the first set of biological fluid samples through a hole array filter, and generating a second set of mass spectra data from a second set of biological fluid samples from a population without the disease after filtration of the second set of biological fluid samples through a hole array filter. The method may also comprise a step of comparing the first and second sets of mass spectra data, wherein a difference between corresponding peaks in the first and second sets of mass spectra data indicates at least one negative marker for detecting the disease. In one embodiment, the structure of the hole array filter through which the first set of samples are filtered and the structure of the hole array filter through which the second set of samples are filtered are substantially identical. In another embodiment, the hole array filter through which the first set of samples are filtered and the hole array filter through which the second set of samples are filtered are separate hole array filters. In another embodiment, each sample is filtered through a separate hole array filter.

Further, one embodiment of the present invention may include a method for detecting a disease in a test subject. The method may comprise a step of utilizing the at least one negative marker detected by the method as explained in the preceding paragraph as a diagnostic marker to detect the disease in the test subject. In one specific embodiment, the method may comprise steps of filtering a biological fluid sample from the test subject through a hole array filter, and generating mass spectra data from the filtered biological fluid to evaluate an amount(s) of the at least one negative marker. In the embodiment, the method may also comprise a step of diagnosing the disease in the test subject based on the amount(s).

These and other objects and advantages of the invention will be apparent from the following description, the accompanying drawings and the appended claims.

### BRIEF DESCRIPTION OF THE DRAWINGS

While the specification concludes with claims particularly pointing out and distinctly claiming the present invention, it is believed the same will be better understood from the following description taken in conjunction with the accompanying drawings, which illustrate, in a non-limiting fashion, the best mode presently contemplated for carrying out the present invention, and in which like reference numerals designate like parts throughout the Figures, wherein:

FIG. 1 is a flow chart illustrating a method according to one embodiment of the present invention.

FIGS. 2-1 to 2-11 are chromatograms of normal pre-filtered and post-filtered sera samples.

FIGS. 2-12 to 2-19 are chromatograms of pre-filtered and post-filtered sera samples known to have lung cancer.

FIG. 3a shows a cross section of a filter for use in the present invention.

FIG. 3b shows a top view of a hole array of the filter.

FIGS. 4a-4g show the steps that may used in making filters in accordance with the instant invention.

FIGS. 5-1 to 5-34 show chromatograms of pre-filtered sera vs. post-filtered sera showing the enhancement of a peak in a chemosensitivity screening assay.

FIGS. 6a-1 to 6a-32 show the chromatograms of pre-filtered sera vs. post-filtered sera of lung cancer patients.

FIGS. 6b-1 to 6b-34 show the chromatograms of pre-filtered sera vs. post-filtered sera of normal patients.

FIGS. 7-1 to 7-42 shows the chromatograms of pre-filtered sera vs. post-filtered sera of pancreatic cancer patients.

FIGS. 8-1 to 8-12 show the chromatograms of pre-filtered urine vs. post-filtered urine of both normal and bladder cancer patients.

FIG. 9 shows steps that may be used in making filters in accordance with the instant invention.

FIGS. 10a to 10e illustrate the results of filtering of serum through a hole array filter according to one embodiment of the present invention.

### DETAILED DESCRIPTION OF THE INVENTION

The present disclosure will now be described more fully with reference to the Figures in which various embodiments of the present invention are shown. The subject matter of this disclosure may, however, be embodied in many different forms and should not be construed as being limited to the embodiments set forth herein.

A method for enhancing mass spectra data is described. For simplicity and illustrative purposes, the principles of the present invention are described by referring to various exemplary embodiments thereof. Although the preferred embodiments of the invention are particularly disclosed herein, one of ordinary skill, in the art will readily recognize that the same principles are equally applicable to, and can be implemented with other compositions and methods, and that any such variation would be within such modifications that do not part from the scope of the present invention. Before explaining the disclosed embodiments of the present invention in detail, it is to be understood that the invention is not limited in its application to the details of any particular embodiment shown, since of course the invention is capable of other embodiments. The terminology used herein is for the purpose of description and not of limitation. Further, although certain methods are described with reference to certain steps that are presented herein in a certain order, in many instances, these steps may be performed in any order as may be appreciated by one skilled in the art, and the methods are not limited to the particular arrangement of steps disclosed herein.

The needs identified in the foregoing Background, and other needs and objects that will become apparent from the following description, are achieved in the present invention, which comprises, but is not limited to, methods for predicting and detecting diseases, and methods for predicting response to the treatment of diseases. The present invention is especially effective for predicting, detecting, and predicting the response to the treatment of diseases such as lung cancer, pancreatic cancer, and bladder cancer, but is in no way limited to those diseases. This is because one of the principles embraced by invention relates to the removal of unwanted substances in the samples which results in better peak generation and cleaner data. As such, the filters and methods of the instant invention are not disease specific.

Mass spectroscopic chromatograms were first compared to find differences between normal fluid and fluid of humans with a certain disease, identified herein as disease A. While chromatograms are illustrated in the present disclosure, one of ordinary skill in the art will realize that the use of, and analysis of, any plot of frequency versus time may be utilized without deviating from the scope and spirit of the present invention. This may include, but is not limited to, spectrograms.

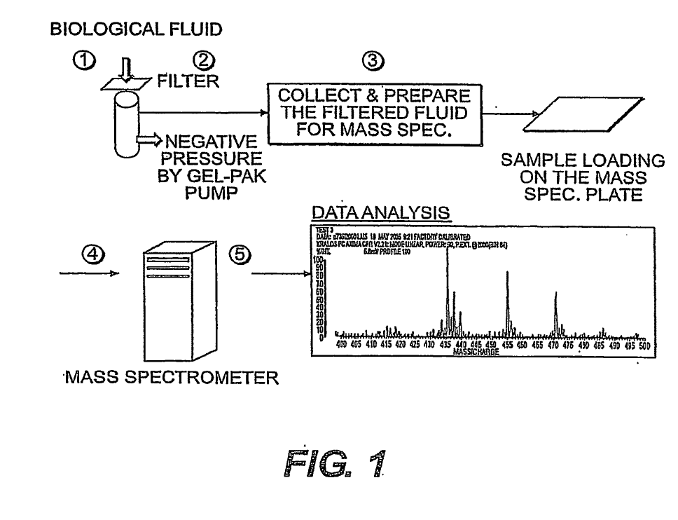

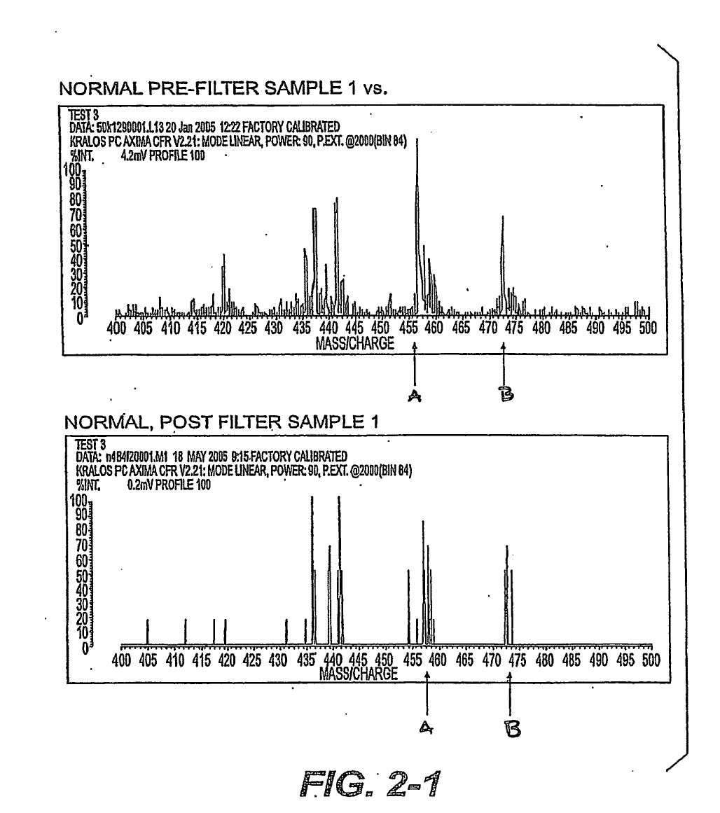

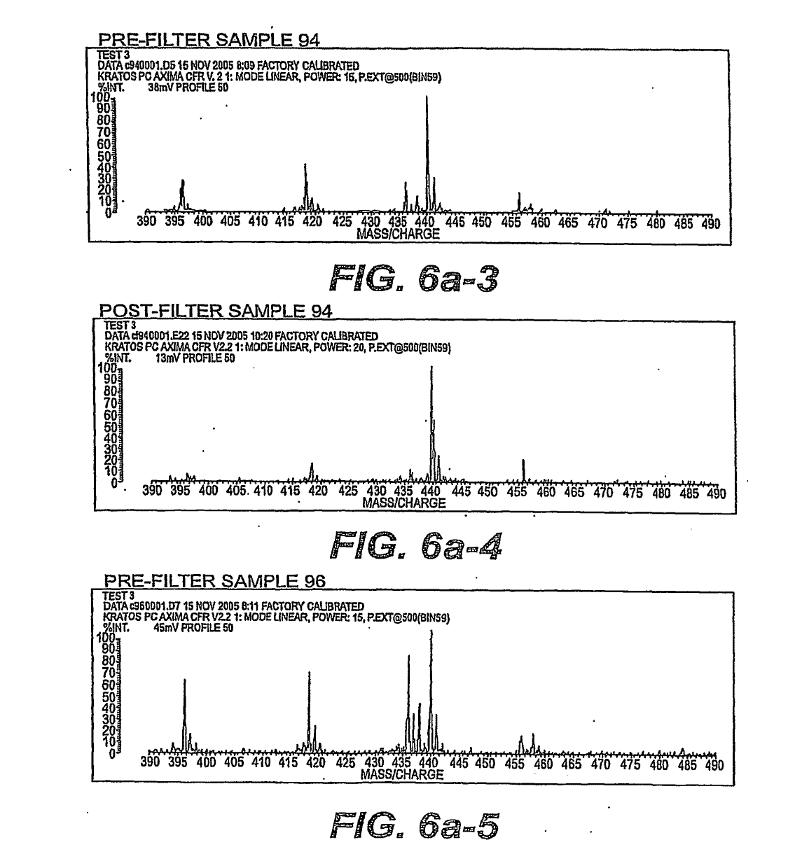

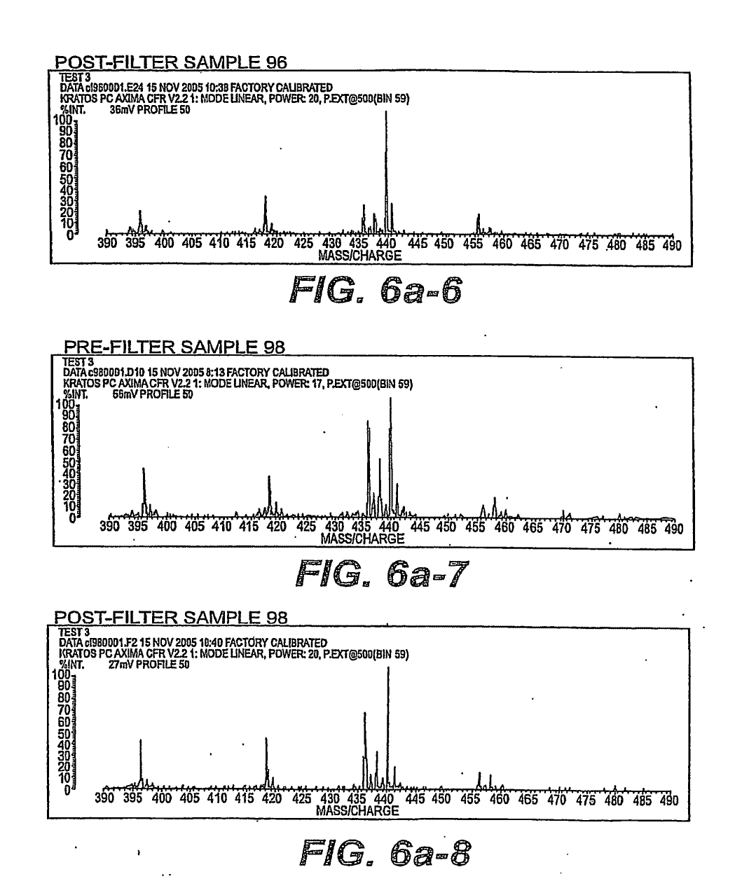

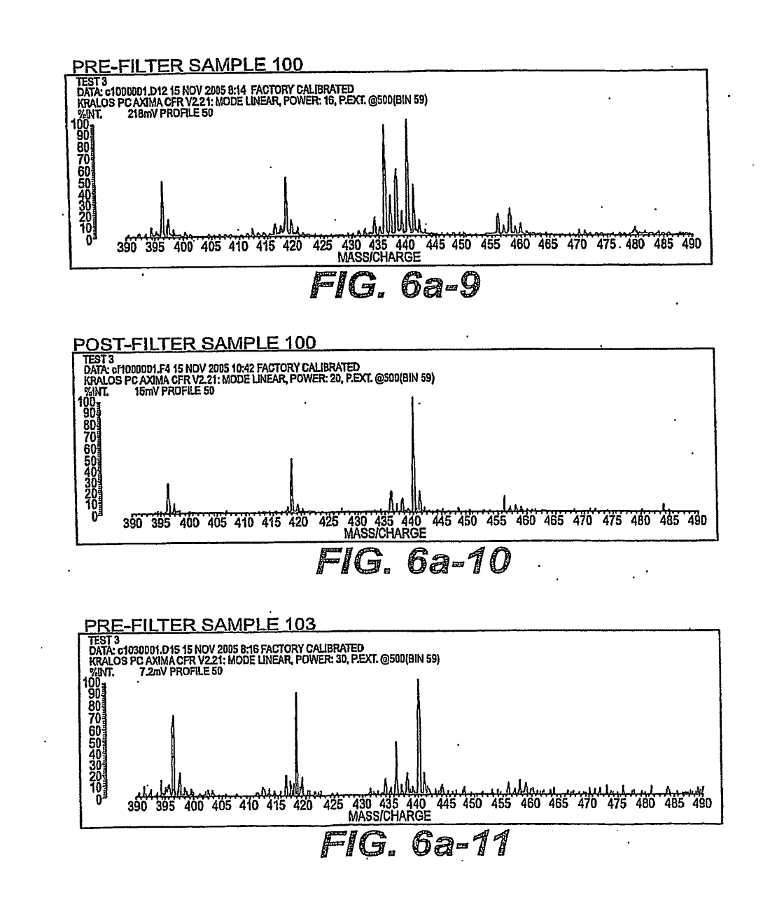

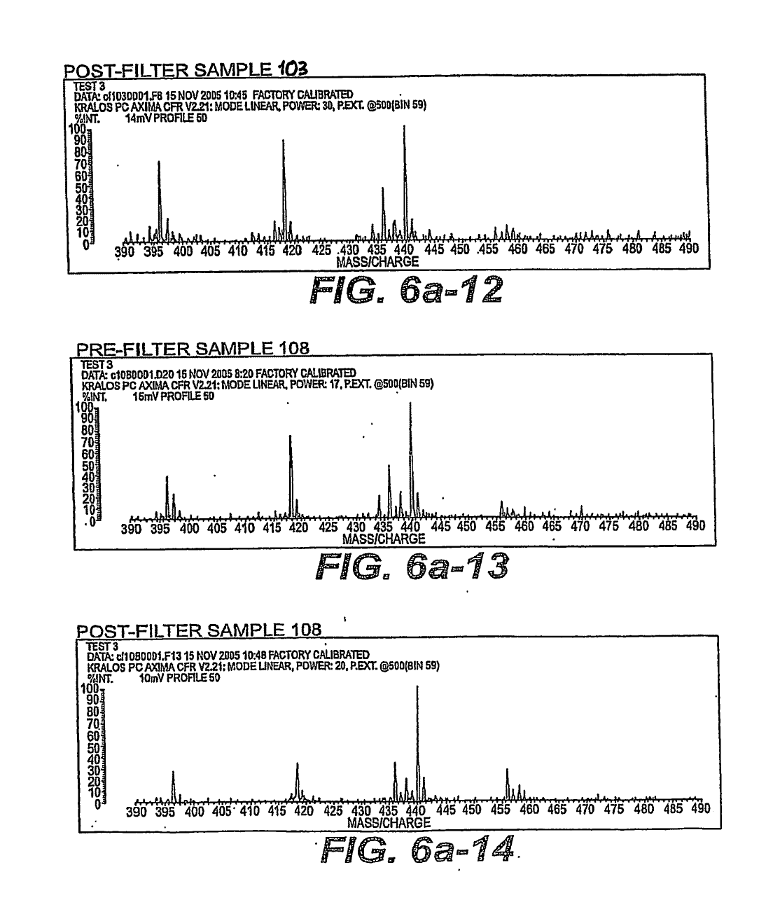

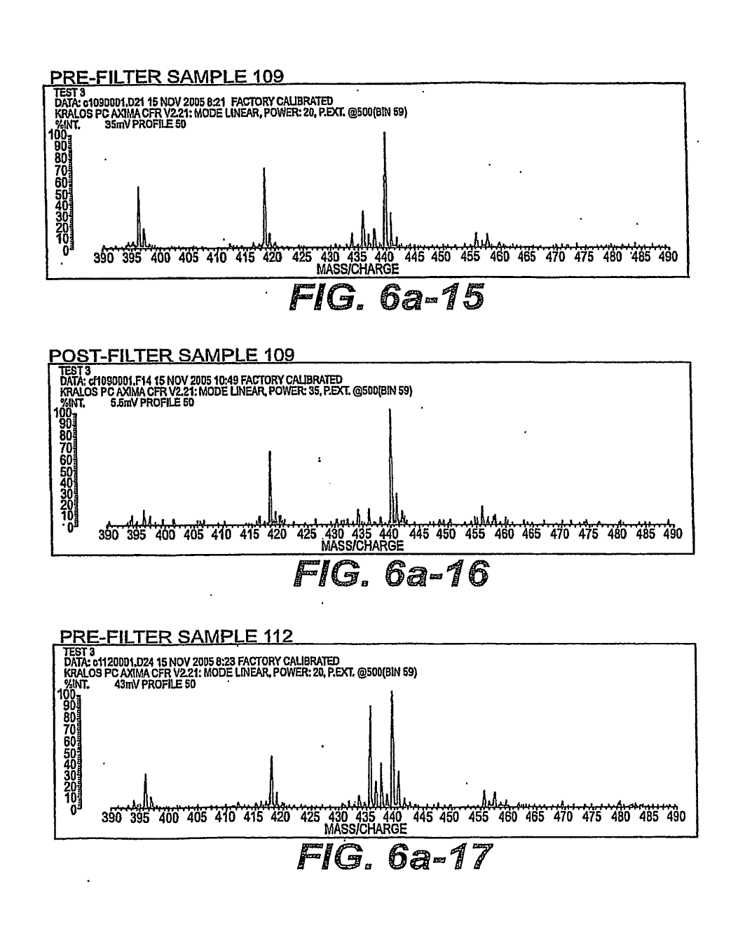

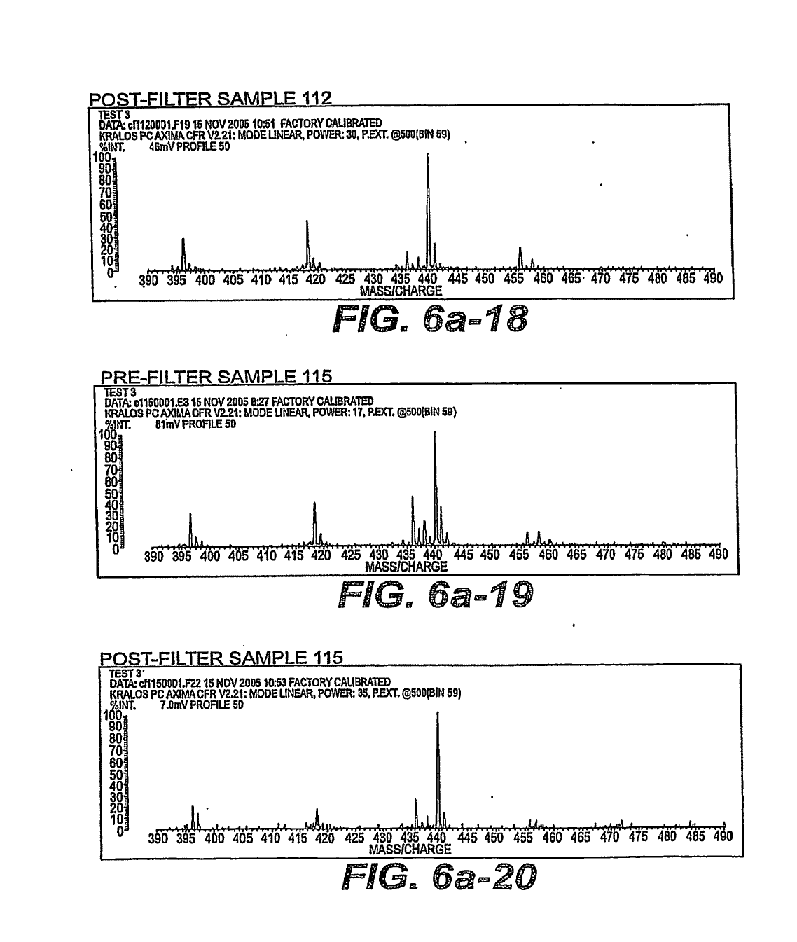

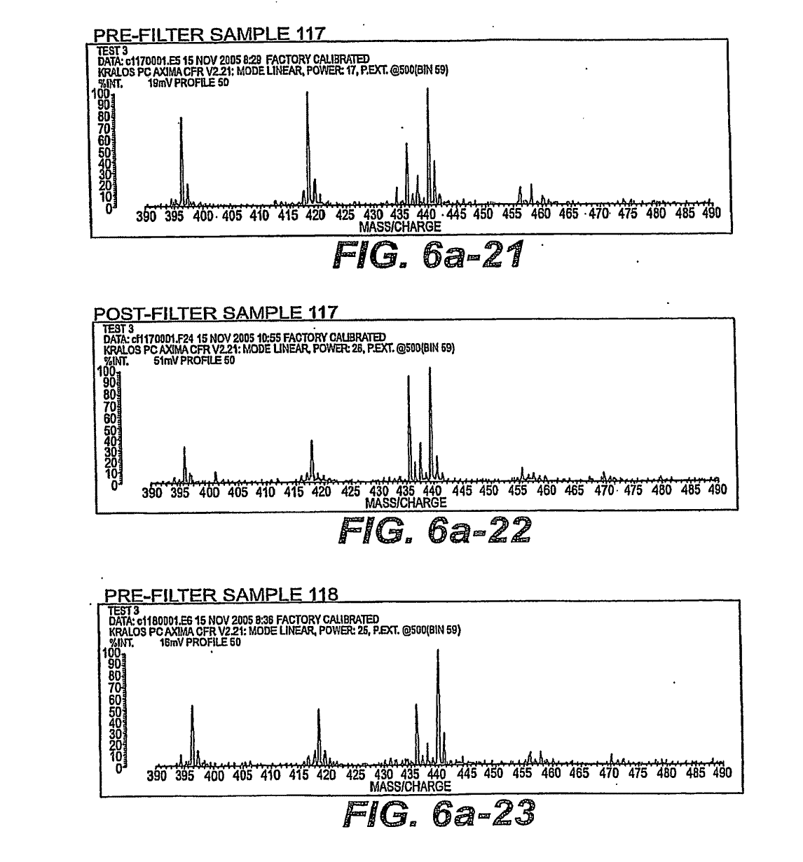

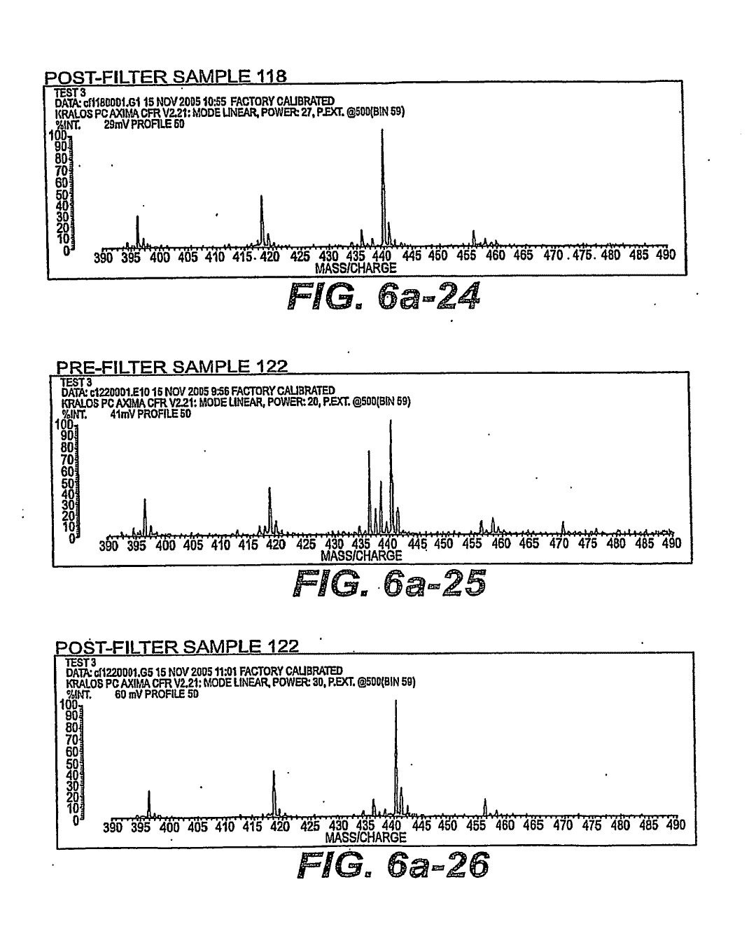

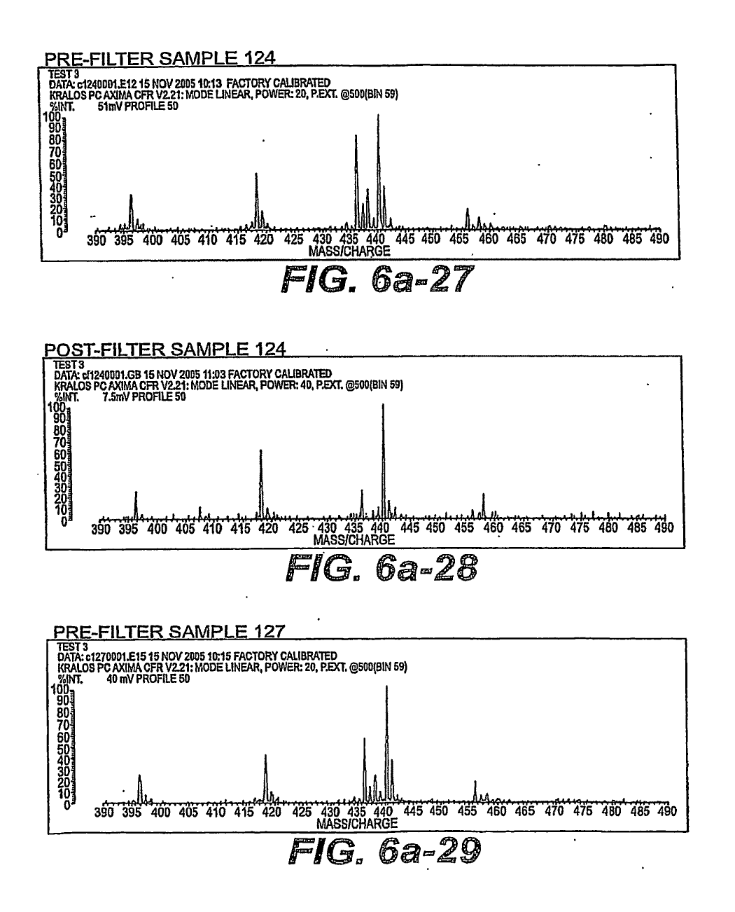

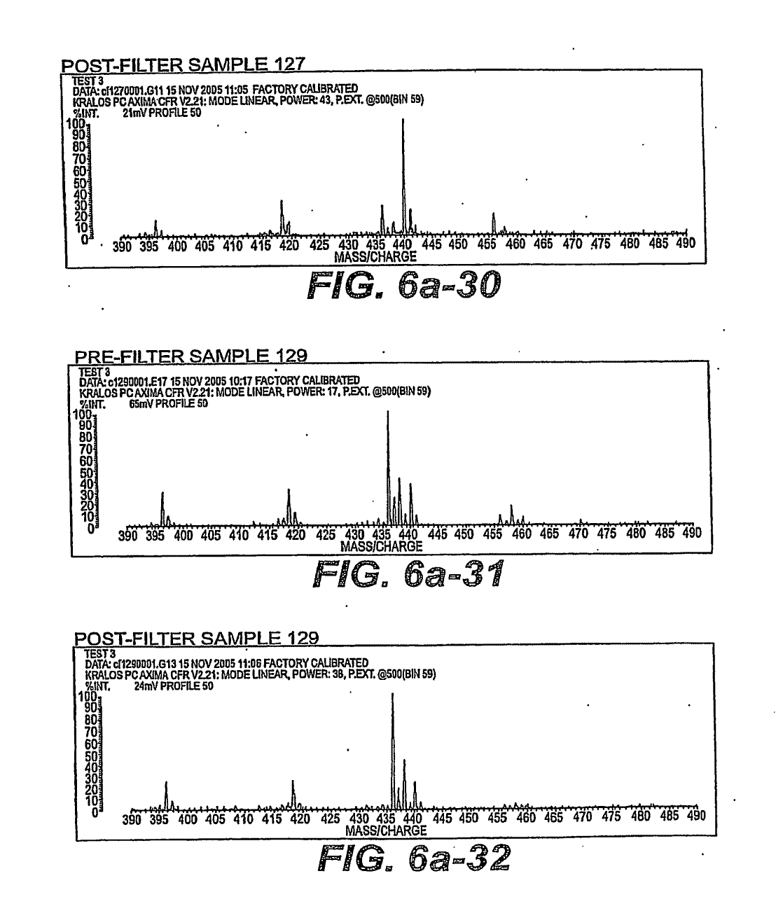

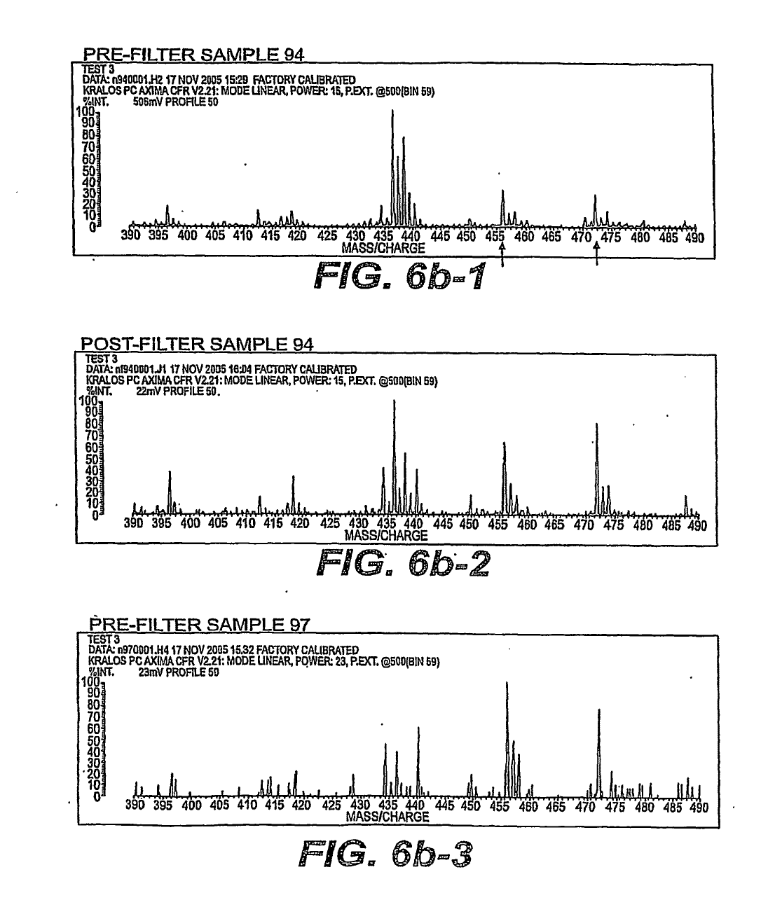

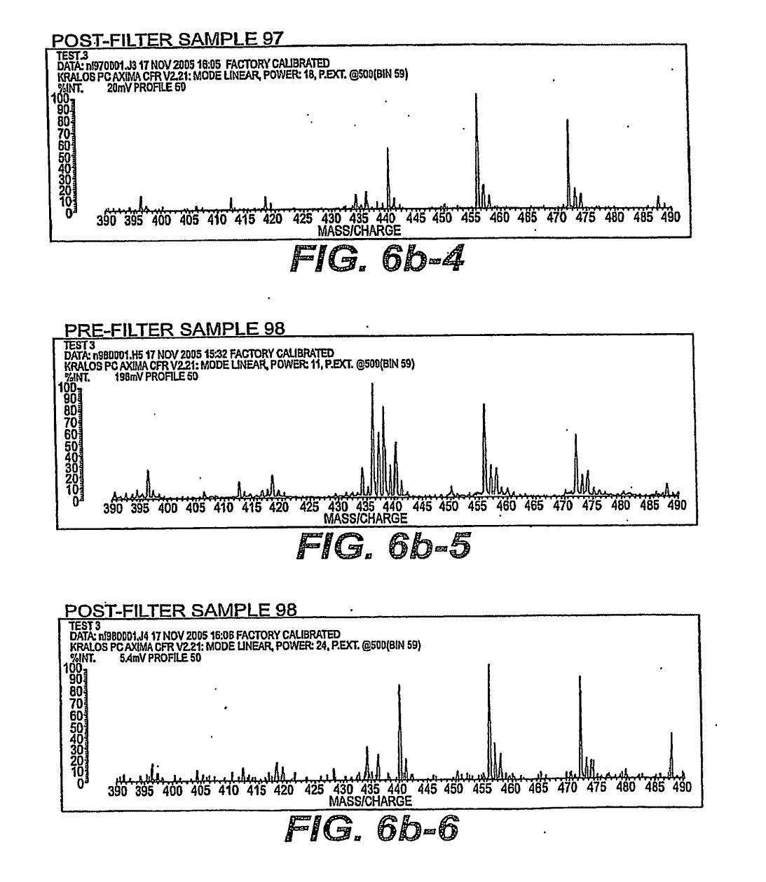

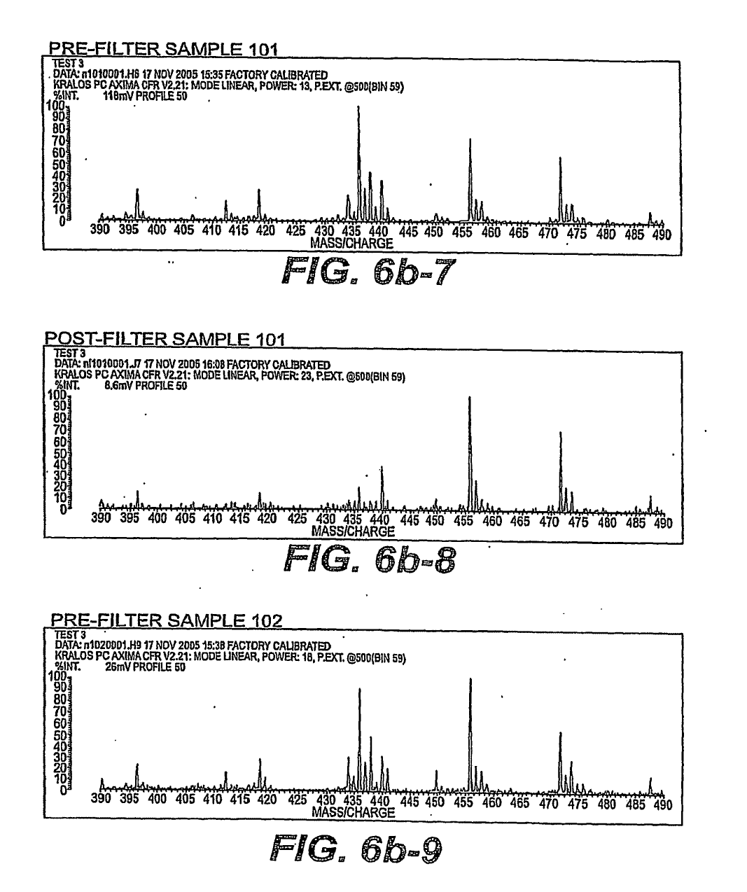

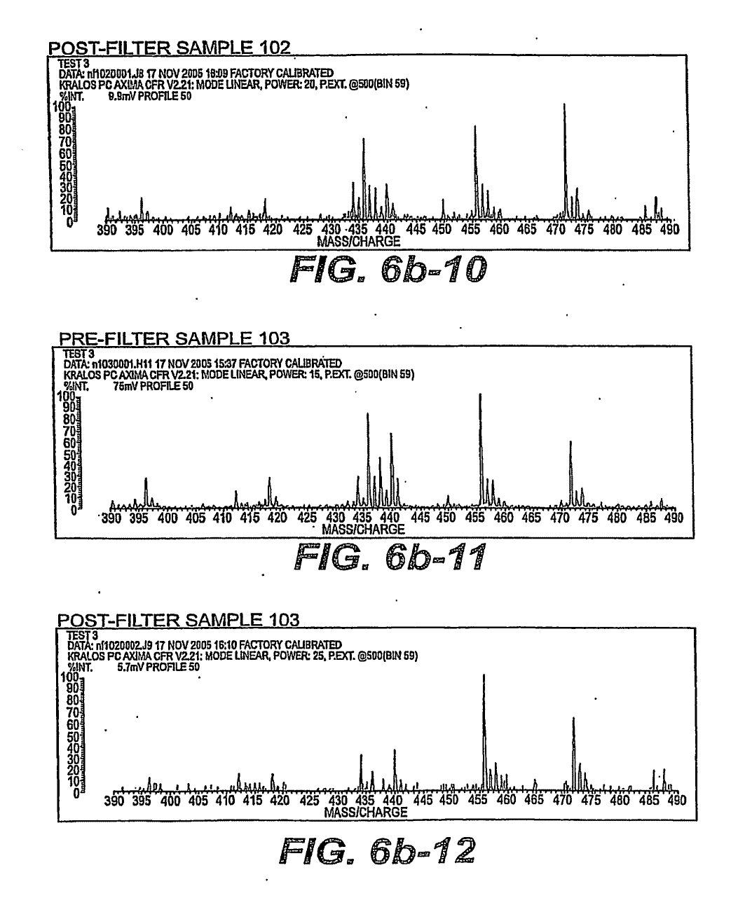

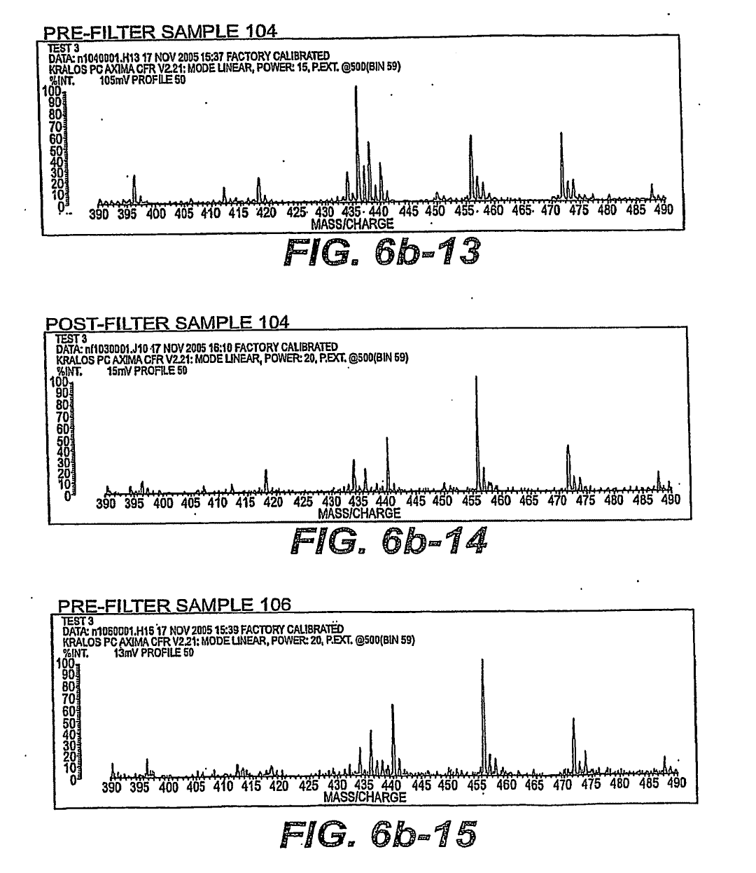

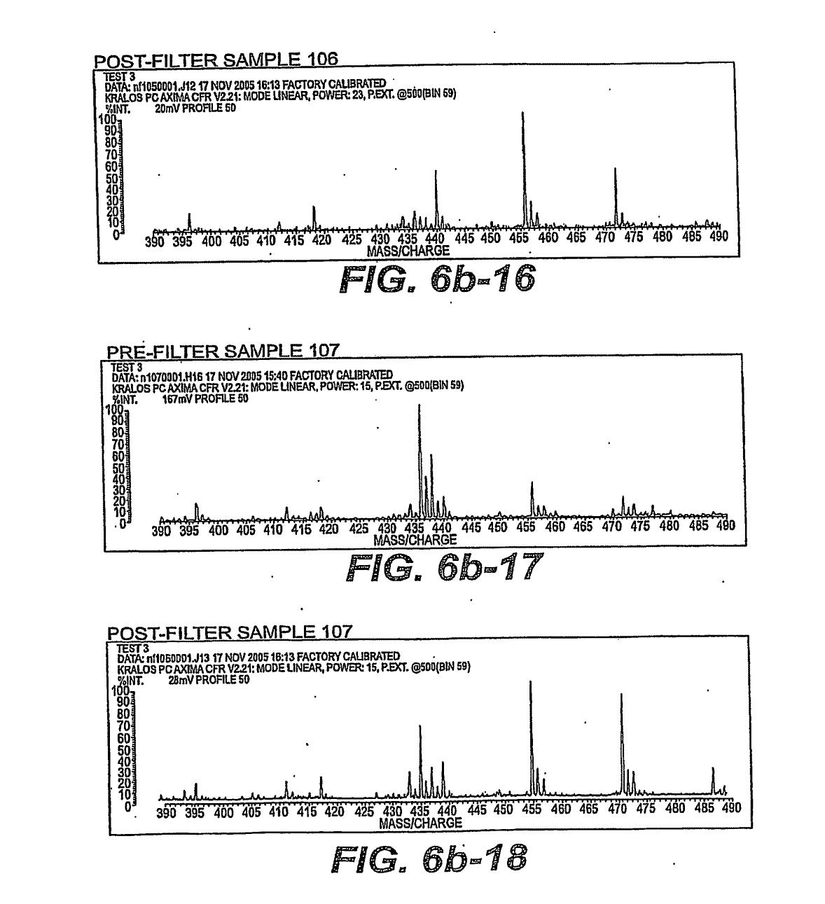

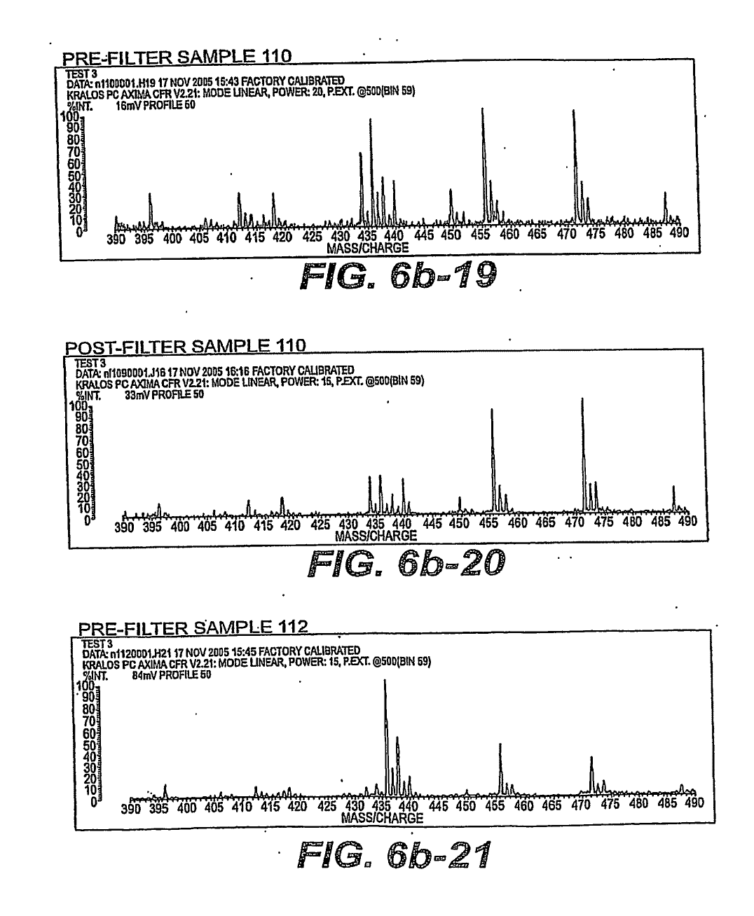

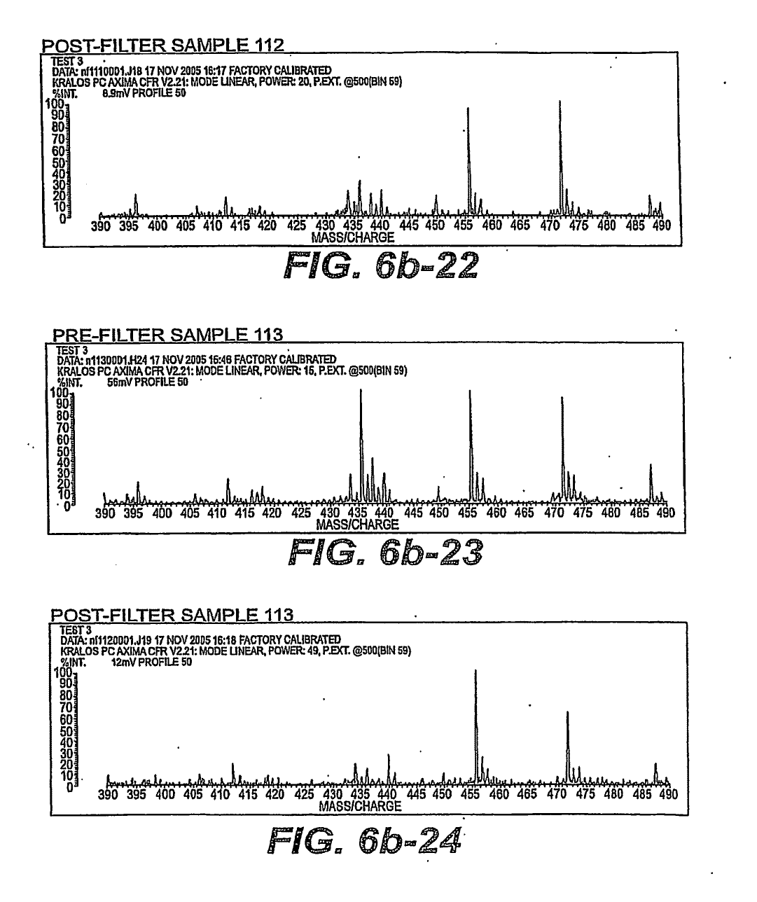

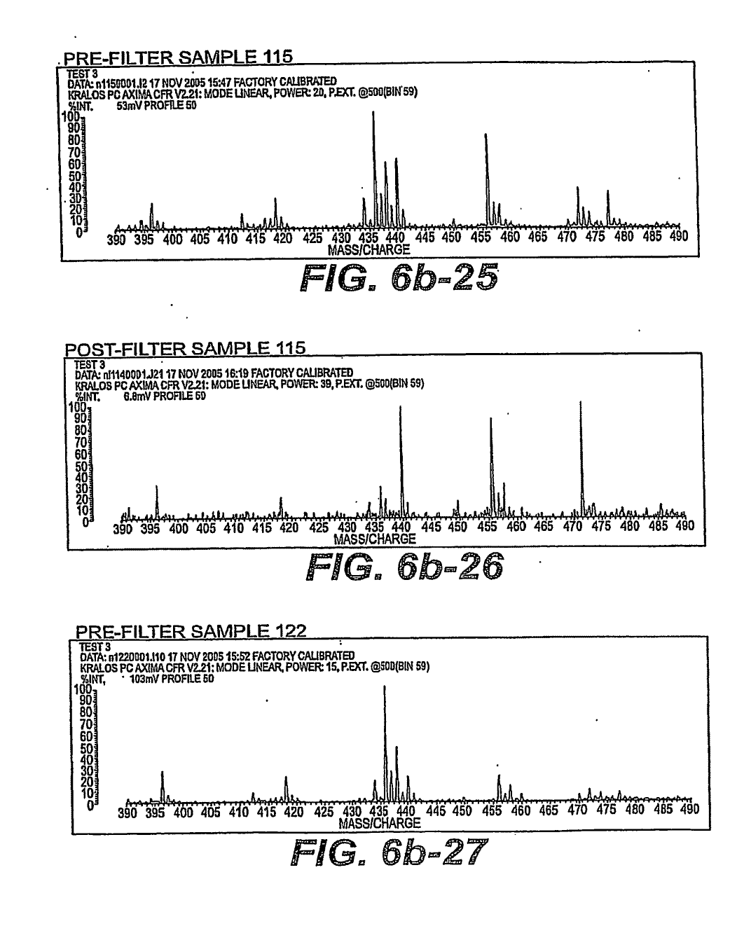

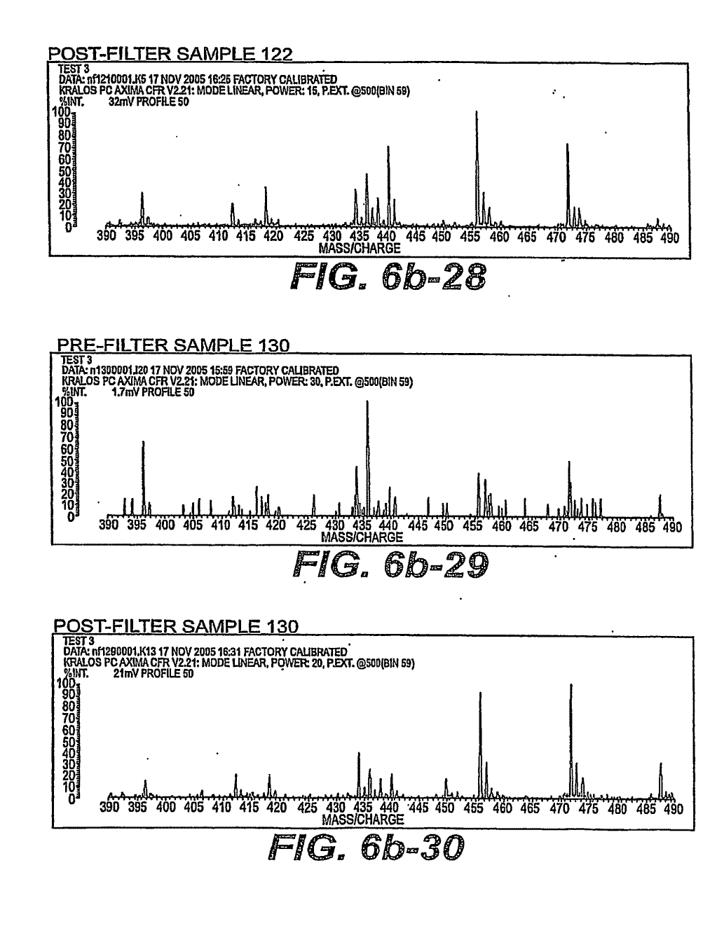

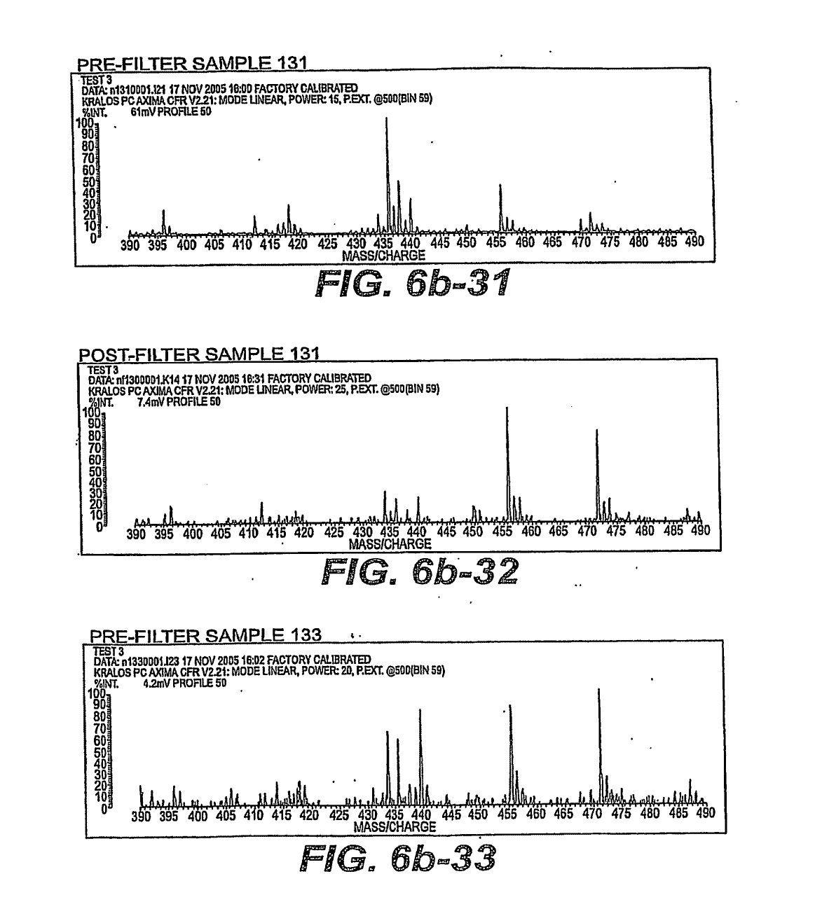

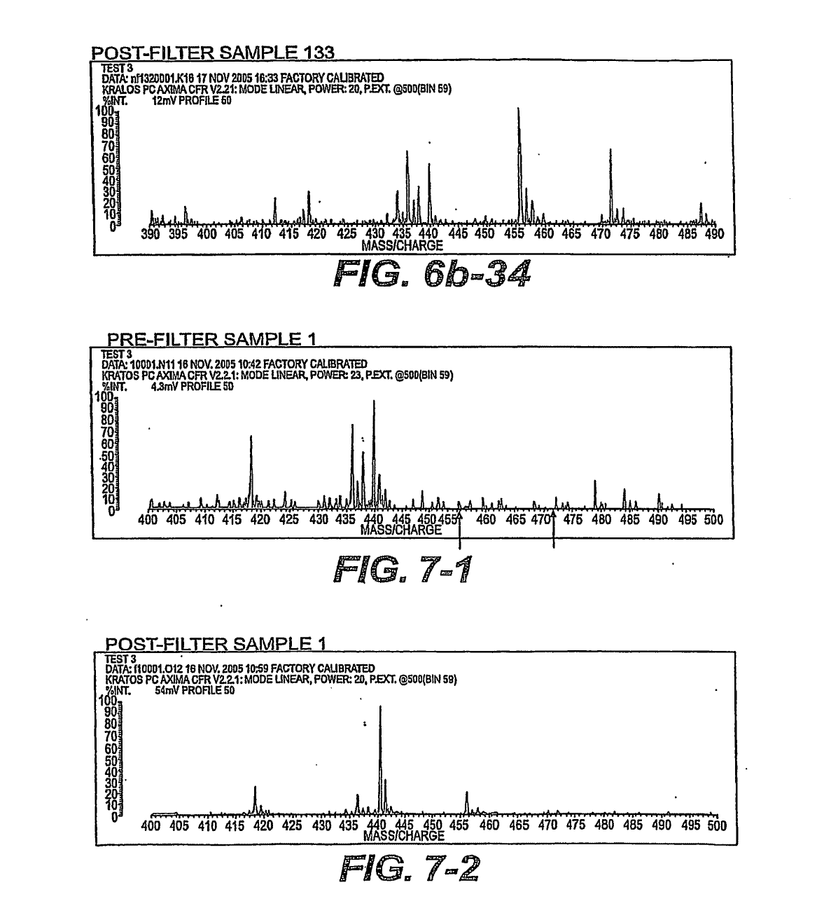

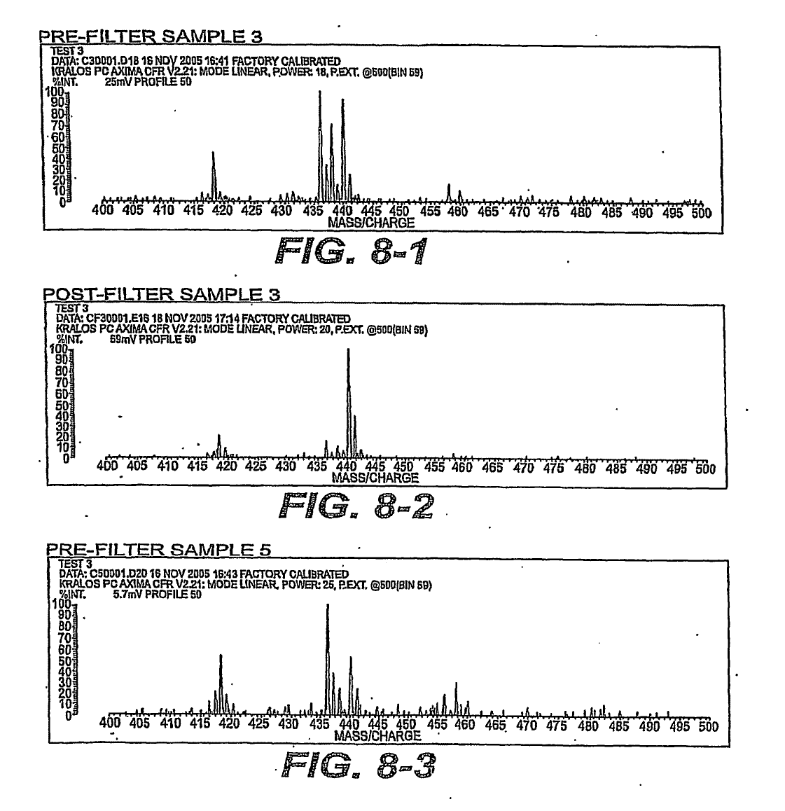

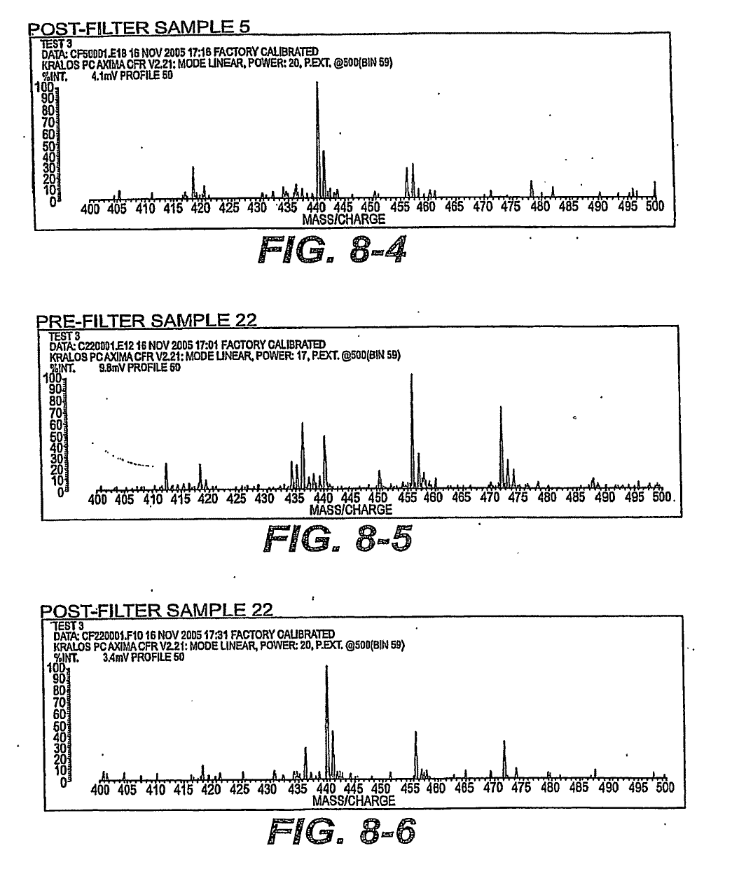

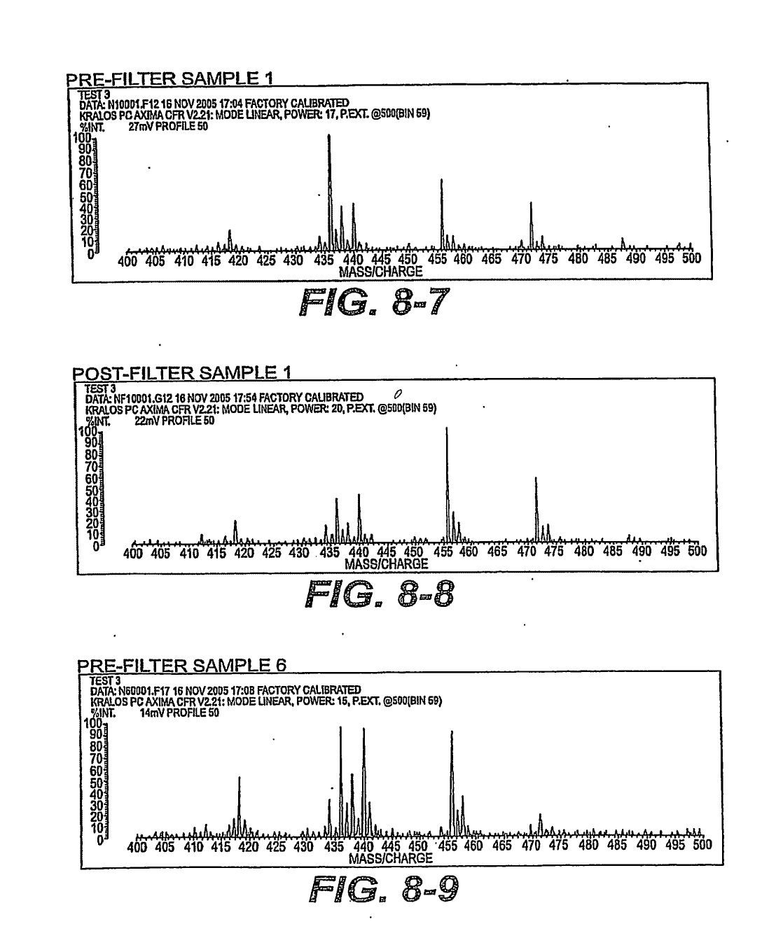

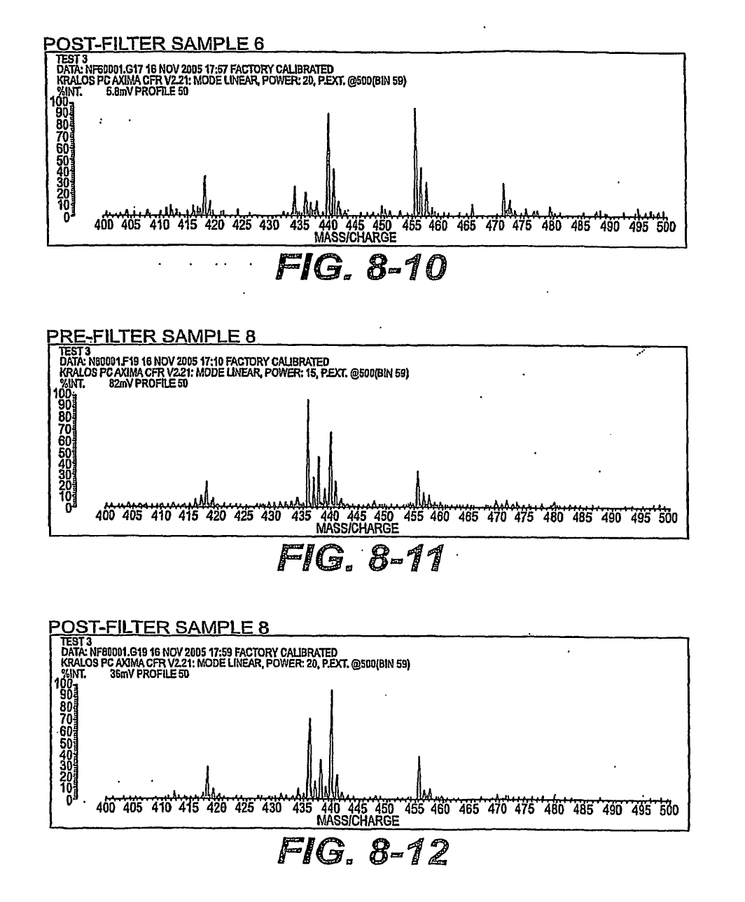

In the present invention, the compared chromatograms focused on a high molecular range because the differences were thought to be not in small molecules but proteins. However, a special peak difference in the high molecular range could not be identified. Accordingly, attempts were made to find differences between two fluid in a low molecular range. Substantial differences were identified at the spots labeled as peaks A and B in FIGS. 2-1 to 2-19 between normal and disease A fluid chromatograms. Normal serum chromatograms show high peaks at the spots corresponding to A and B, but disease A fluid chromatograms do not show any peak or show only small peaks at those spots.

In another aspect of the invention, mass spectroscopic chromatograms were compared between groups responding to a particular chemotherapy treatment with those that did not respond to that particular chemotherapy treatment. A peak was identified in a substantial number of the non-responders. These results can be seen in FIGS. 5-1 to 5-34.

While the data generated by the above assays proved useful, it was determined that the data could be improved. Surprisingly, it was determined that a purification step of biological fluid enhances the ability to detect the presence or absence of peaks indicating biomarkers. Hole array filters were used to purify the serum. As a result of the filtration, and as discussed herein, sensitivity increased by 10% and specificity increased by 25% after use of the filter according to the present invention.

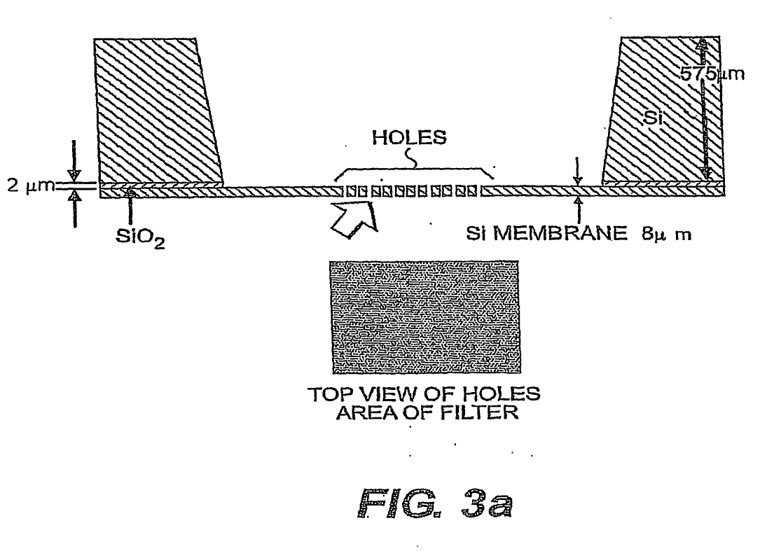

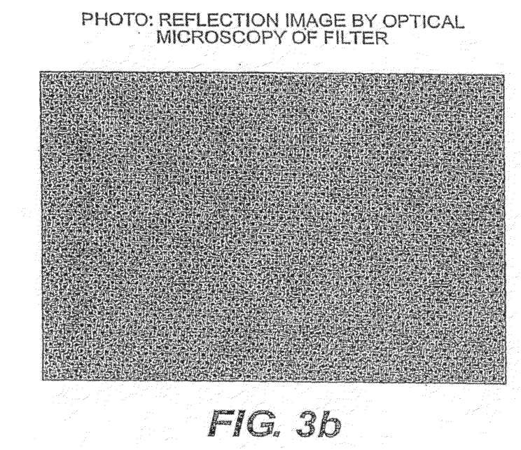

The filters for use in the present invention may comprise an array of holes formed in a silicon membrane of about 3 to 20 μm in thickness. Preferably, the membrane thickness is between about 6-10 μm. If the thickness is less than 6 μm, the hole array area may become very fragile. If the thickness is more than 10 μm, filtration time may increase due to the resistance of the hole surface area A. Thickness of more than 10 μm may also increase the difficulty of making smooth holes. The size of the hole array may be between 1 mm by 1 mm and 10 mm by 10 mm. lithe area is smaller than 1 mm by 1 mm, the amount of filtered biological fluid may not be enough to generate adequate data. lithe area is larger than 10 mm by 10 mm, the amount of biological fluid may become too much and the filter may become more expensive. The size of the holes in the array may be from about 1 μm to 20 μm, and preferably about 1-10 μm. In this instance, the term “size” refers to diameter for a circular hole and diagonal for a square hole. If the size is smaller than 1 μm, the filter hole array area may break when negative pressure is applied. If the size is larger than 10 μm, unwanted compounds of biological fluid may tend to go through the filter holes and the filtration process may become insufficient. The hole pitch, or distance between holes, may be about three times the size of the boles (preferably 3-30 μm) but may be more or less than three times the hole size depending on the particular application (see FIGS. 3a and 3b).

Hole array filters according to the present invention may consist of mainly two areas—a thin area with a hole array, and a thick outer area to improve filter rigidity. The material of the filter may be rigid and may be easily processed to precise designed patterns, as one of ordinary skill in the art will realize. Filter materials may include, but are not limited to, materials such as metal or semiconductor material. One example is Si(thick layer)/SiO2/Si(thin layer with hole array). If Si(thick layer) is tapered toward Si(thin layer with hole array), the flow of biological fluid through the hole array filter becomes smoother. Another material that may be used for hole array filters is Ni/Cu. The hole array filter material should be rigid and with evenly made holes matching the designed size.

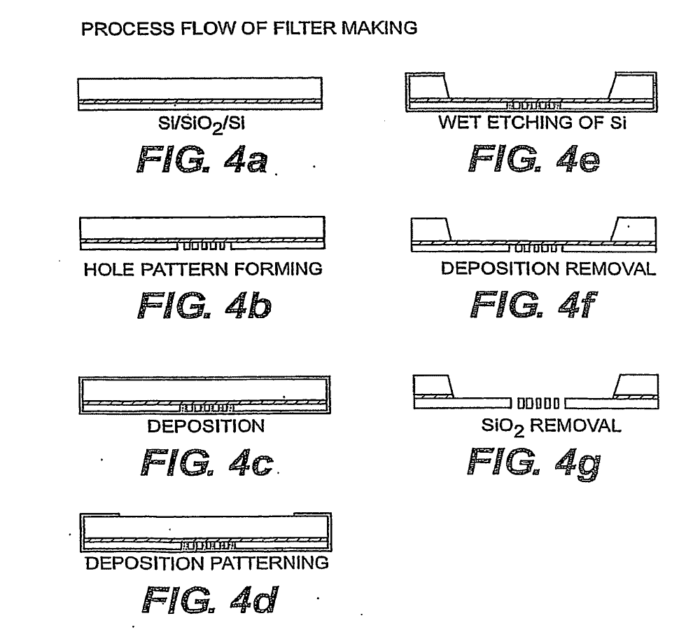

The filters used in the present invention may be made by any method known in the art of lithography or filter making. In one exemplary method, a silicon substrate of about 575 μm thickness may be used as the starting material. A thin layer of silicon dioxide may then be formed on one side of the substrate using any common method such as chemical vapor deposition (CVD), or through further oxidation of the surface portion of the substrate by exposure to an oxygen containing plasma. The silicon dioxide layer may be about 2 μm thick. A thin layer of silicon may be formed on the oxide layer by any method such as CVD or thin film crystallization (see FIG. 4A). The substrate may then be flipped over so the thin crystalline silicon film is on the bottom or backside of the substrate. This silicon naturally develops a very thin layer of silicon dioxide of a thickness on the order of a few Angstroms. This substrate is typically called Silicon On Isolator (SOI) substrate.

The resist material may be coated on the SOI surface. Resist material can be photoresist for photo exposure such as Ultra Violet light and electron beam resist for electron beam exposure at the following processes. Patterned mask may then be applied onto, or in proximity to, the resist. Next, ionizing radiation such as ultra violet light or electron beam may be applied to the resist through the patterned mask. After the mask is removed, unnecessary pattern portion of the resist may be removed by removing material such as solvent. Finally, an Si layer may be etched using either a dry or a wet process to make a certain shaped hole array, as shown in FIG. 4B, after removing the whole resist. In such cases, silicon dioxide layer works as etching stopping layer.

A protective layer may then be applied over the entire substrate including over the hole array (FIG. 4C). A portion of the protective layer on the top side of the substrate and symmetrically arranged compared to the underlying hole array but wider than the hole may then be removed through a mask and resist etching process (FIG. 4D). A wet etch of the exposed substrate may then be performed until the oxide layer is reached resulting in the exposure of the oxide layer surrounding the underlying hole array and tapered walls of the side of the exposed silicon substrate, as shown in FIG. 4E. The remainder of the protective layer may then be removed by a wet or dry etching process as shown in FIG. 4F. The exposed portion of the oxide layer may then be removed by a wet or dry etching process resulting in a finished filter as shown in FIG. 4G.

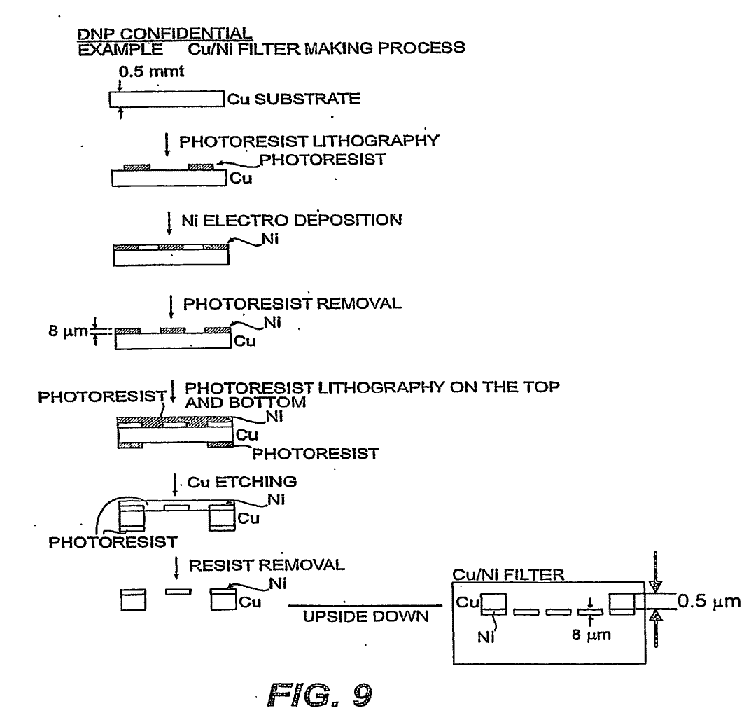

Filters in accordance with the instant invention may also be made with other materials including, but not limited to, Ni/Cu. As one of ordinary skill in the art will realize, the steps used to form such filters will be similar to those above and shown in FIG. 9.

Although specific steps and processes have been used to describe the formation of the filters used in the present invention, these steps and processes are exemplary only. As is well known in the art, any processes may be used to form a hole array in a thin layer of silicon. Additionally, the thicknesses of the different layers and sizes of the holes and distances between the holes are provided as exemplary only and are not meant to be limiting in any manner. Additionally, the word “hole” is not meant to be limited to a void of any particular shape but may be round, square, triangle, or any other shape. As such, cross sectioning of the holes need not be cylindrical in shape. Further, the filter material is not limited to silicon as the filter may comprise any common filter material.

It should also be noted that any suitable biological samples may be used in embodiments of the invention. Biological samples include tissue (e.g., from biopsies), blood, serum, plasma, nipple aspirate, urine, tears, saliva, cells, soft and hard tissues, organs, semen, feces, and the like. The biological samples may be obtained from any suitable organism including eukaryotic, prokaryotic, or viral organisms.

The biological samples may include biological molecules including macromolecules such as polypeptides, proteins, nucleic acids, enzymes, DNA, RNA, polynucleotides, oligonucleotides, carbohydrates, oligosaccharides, polysaccharides;

fragments of biological macromolecules set forth above, such as nucleic acid fragments, peptide fragments, and protein fragments; complexes of biological macromolecules set forth above, such as nucleic acid complexes, protein-DNA complexes, receptor-ligand complexes, enzyme-substrate, enzyme inhibitors, peptide complexes, protein complexes, carbohydrate complexes, and polysaccharide complexes; small biological molecules such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphospho-sugars, synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents; and synthetic polymers, oligomers, and copolymers. Any suitable mixture or combination of the substances specifically recited above may also be included in the biological samples.

In order to more fully optimize and characterize the present filtration methods, the hole array filters identified in the following table were evaluated for their ability to cleanse sample and thereby improve the sensitivity and specificity of the present methods.

| Filter Name | Designed hole diameter (μm) | Designed pitch (μm) |
|---|---|---|
| 1-11P | 1 | 11 |
| 5-10P | 5 | 10 |
| 5-20P | 5 | 20 |
| 5-55P | 5 | 55 |
| 10-40P | 10 | 40 |
| 10-110P | 10 | 110 |

Serum samples were filtered with each of the above filters. Evaluation by MALDI-TOF-MS found the following trend in cleansing effect: 1-11P>5-10P>5-20P >5-55P>10-40P>10-110P. That is, filter 1-11P hid the greatest cleansing effect for the samples tested. However, each progressive level of filtration increased the ability to identify certain peaks representing mass ion species as compared to the prior level of filtration. Thus, the mass spectra data for samples filtered with the filters had an improved resolution, or enhanced signal to noise ratio, for potential peaks of interest (e.g., spectral peaks corresponding to the mass to charge ratio of different molecules) compared to unfiltered samples.

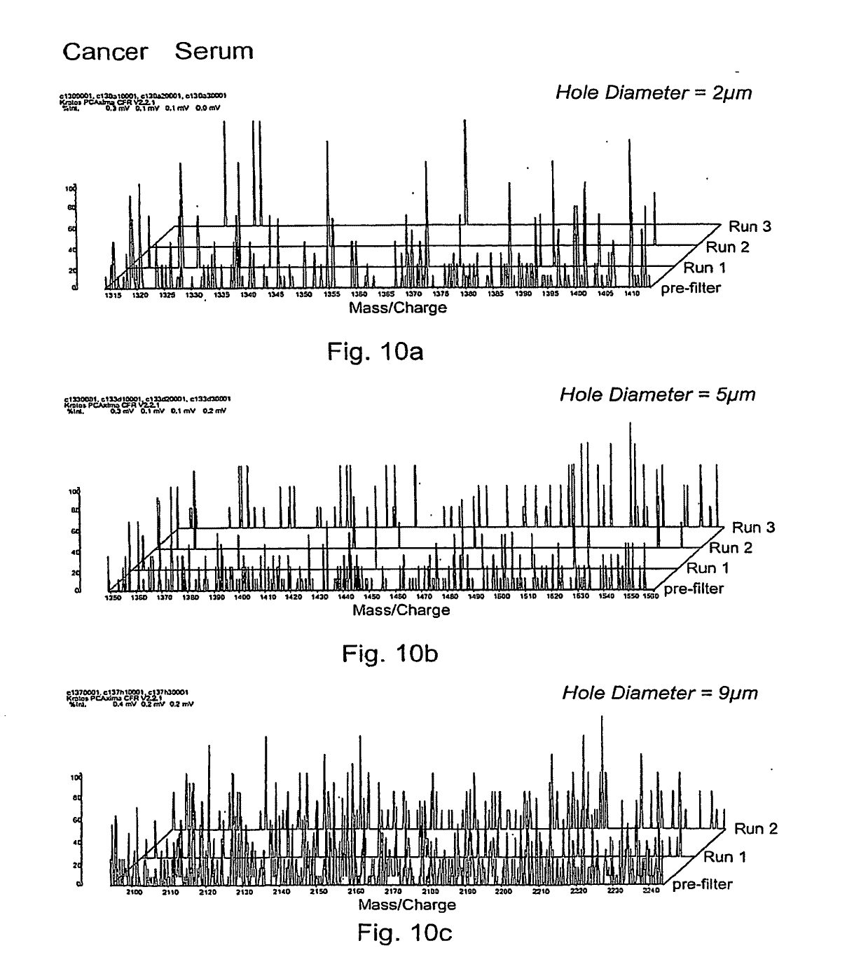

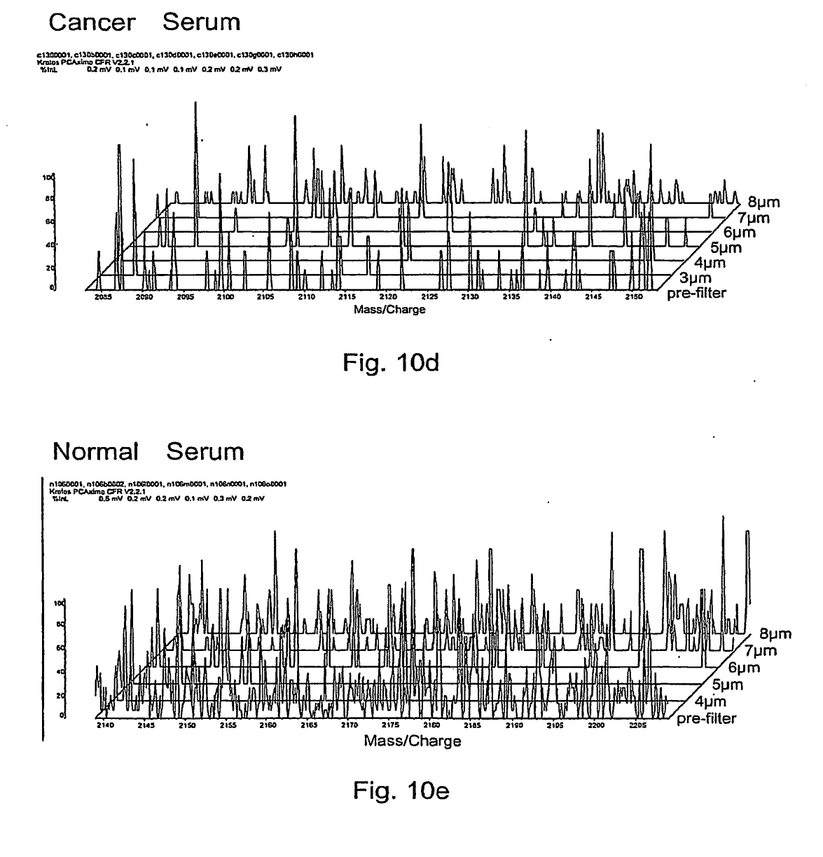

Hole array filters having a hole diameter of 2 μm, 3 μm, 4 μm, 5 μm, 6 μm, 7 μm, 8 μm and 9 μm were also evaluated for cleansing effect. The results of these experimentations are shown in FIGS. 10a to 10e. FIG. 10a is a chromatogram from a sample of cancer serum filtered through a hole array filter with a diameter of 2 μm. FIG. 10b is a chromatogram from a sample of cancer serum filtered through a hole array filter with a diameter of 5 μm. FIG. 10c is a chromatogram from a sample of cancer serum filtered through a hole array filter with a diameter of 9 μm. FIG. 10d is a chromatogram from samples of cancer serum filtered through hole array filters with diameters of 3 μm, 4 μm, 5 μm, 6 μm, 7 μm, and 8 μm. FIG. 10e is a chromatogram from samples of normal serum filtered through hole array filters with diameters of 3 μm, 4 μm, 5 μm, 6 μm, 7 μm, and 8 μm. Some of the 2 μm filters were broken when negative pressure was applied during the filtering process. This is likely due to small holes which cause more pressure in the filter layer, when the filter layer is covered by biological fluid. The remainder of the 2 μm filters, however, improved the signal to noise ratio of the samples.

The results from the 9 μm hole filters tend to give less filtering effect than the smaller hole filters. In some cases, this may be because the larger holes allow an increased amount of those substances (e.g., molecules) in the biological fluid that reduce the signal to noise ratio to pass through the filter. However, in other cases, the larger hole filters may be sufficient to filter out enough of noise-causing substances to increase the resolution of potential peaks of interest. Nonetheless, this evaluation indicated that a filter hole size of between 2 and 9 μm work well because enough of the noise-causing substances were filtered to increase the relevant peaks in the final data.

The following examples 1-4 illustrate specific testing and analysis of sera using the methods and apparatus of the present invention. Each example utilizes the method illustrated in FIG. 1. As shown in FIG. 1, a biological fluid 1 may be filtered through a filter 2, with the resulting sample being collected and prepared for mass spectroscopy 3. The sample may then be loaded onto a mass spectroscopy plate 4, and the samples may be run through a mass spectrometer 5. The mass spectrometry data may then be analyzed as discussed in the examples below. One of ordinary skill in the art will realize that, while each of these examples is specific to a particular disease and testing situation, they are only being provided for illustrative purposes and are not meant to limit the scope and applicability of the present invention.

### EXAMPLE 1

The discussion below discusses a specific lung cancer screening performed using the apparatus and methods according to the present invention discussed above. MALDI-TOF-MS was used to generate a spectra sample data set representing distinct protein/peptide patterns in serum. In one clinical investigation, sera either from patients with lung cancer or healthy controls were obtained before surgical procedures. All final diagnoses were confirmed by histopathology and all controls were at high risk for lung cancer, but without evidence of lung cancer based on clinical presentation and CT scan examination.

The sera were prepared for evaluation by a mass spectrometer by making a matrix of serum samples. The mass spectrometer matrix contained saturated alpha-cyano-4-hydroxycinnamic acid in 50% acetonitrile-0.05% trifluoroacetic acid (TFA). The sera were diluted 1:1000 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used The instrument was set to the following specifications: tuner mode, linear; mass range, 0 to about 5,000; laser power, 90; profile, 100; shots per spot, 5. The output of the mass spectrometer was stored in computer storage in the form of a sample data set.

Before applying the filter, the serum was diluted 1:10 in 0.1% n-Octyl β-D-Glucopyranoside. The micro tubes were cut individually from Micro Amp 8 strip tubes. A hole was made in the bottom part of the micro tube by using a needle (Becton Dickinson 20G1) having a diameter of 0.9 mm or less. The micro tube with hole was placed on the metal plate of a gel-pak suction apparatus (air pump) and the hole was adjusted to the same air-flow direction of the air pump. The filter was placed on the top of the micro tube. 20 μl of serum was loaded on the upper part of filter. The serum solution spread out, filling the inner square of the filter. The negative air flow was applied by pumping the air pump manually. The dropped sera solutions from the filter to the micro tube were collected and transferred to the new tube. The filtered serum was further diluted 1:100 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

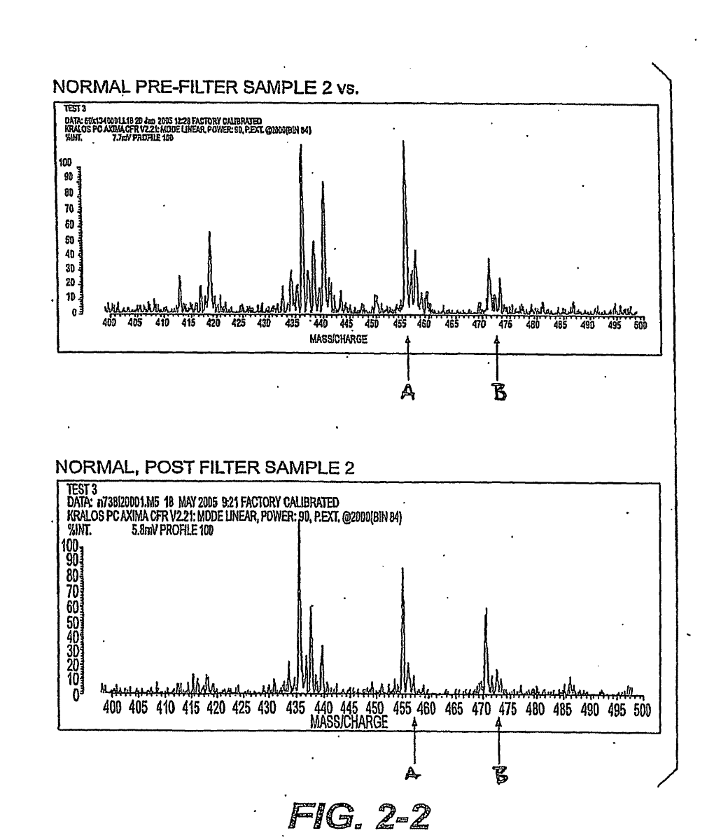

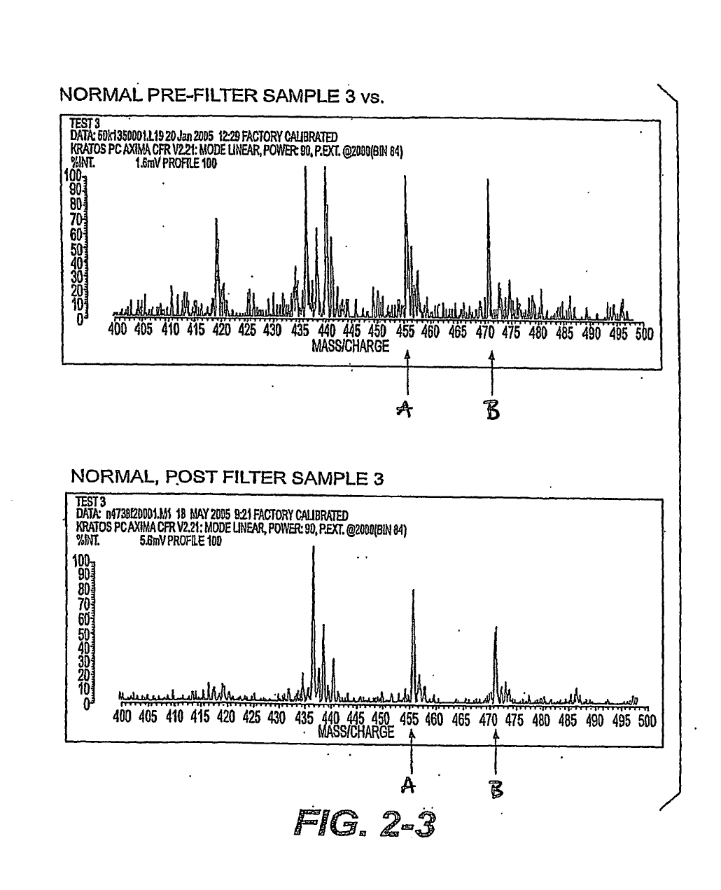

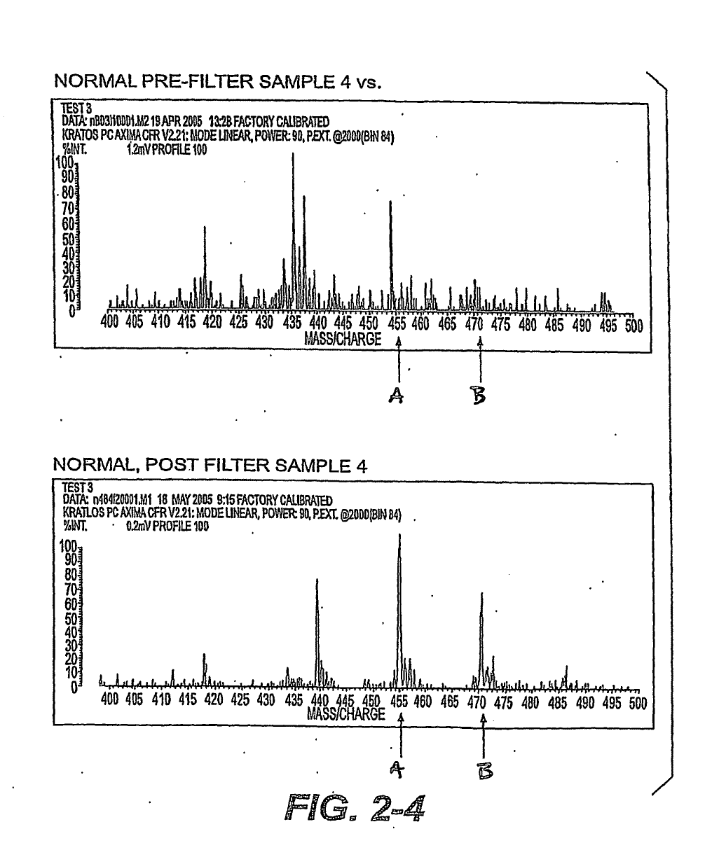

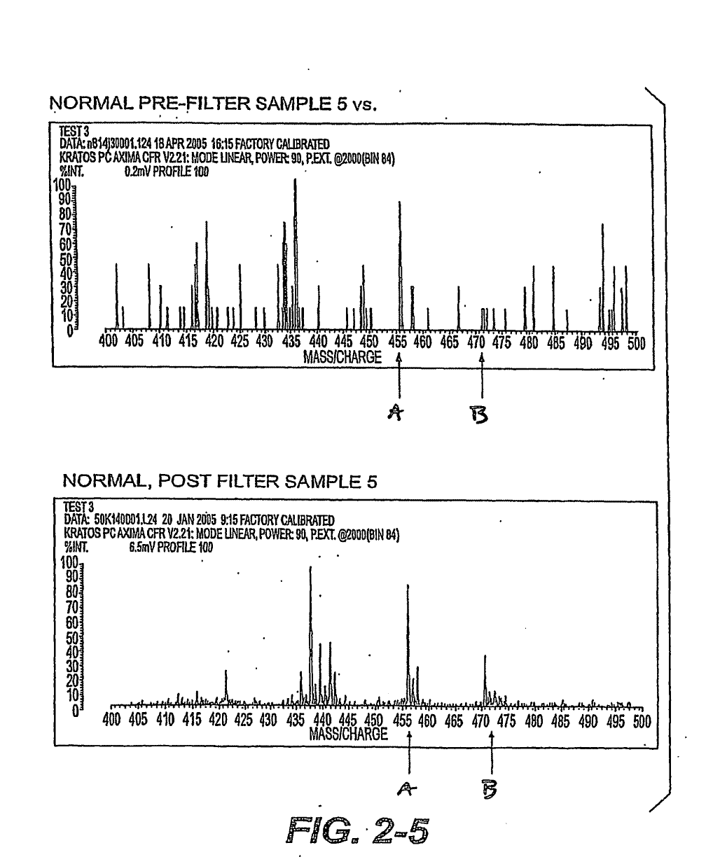

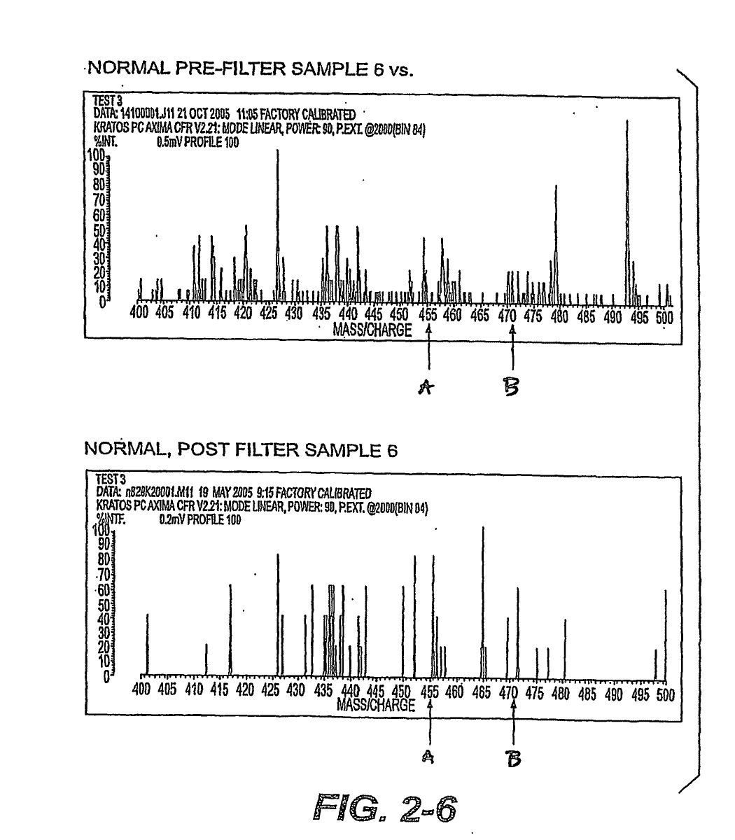

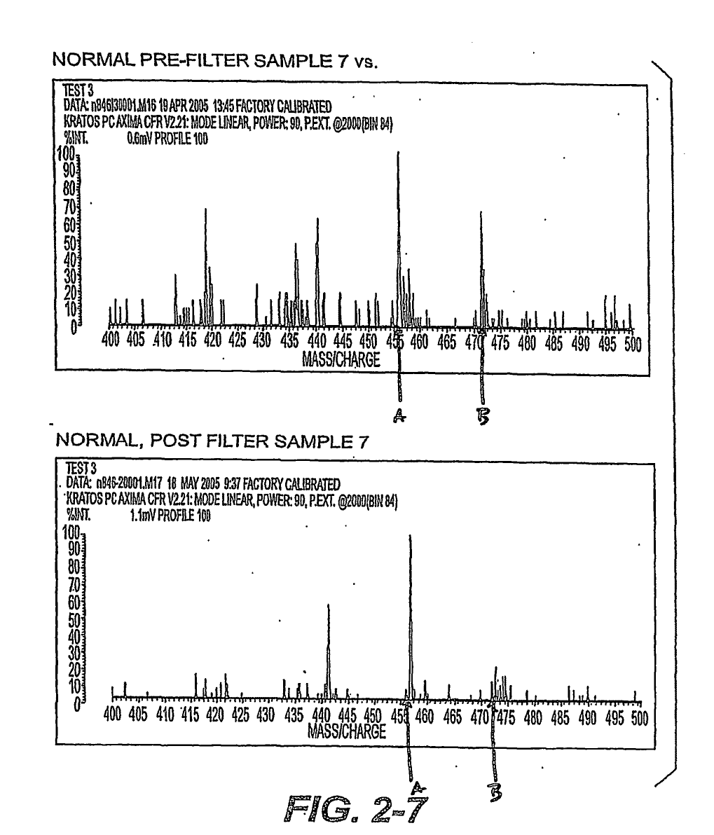

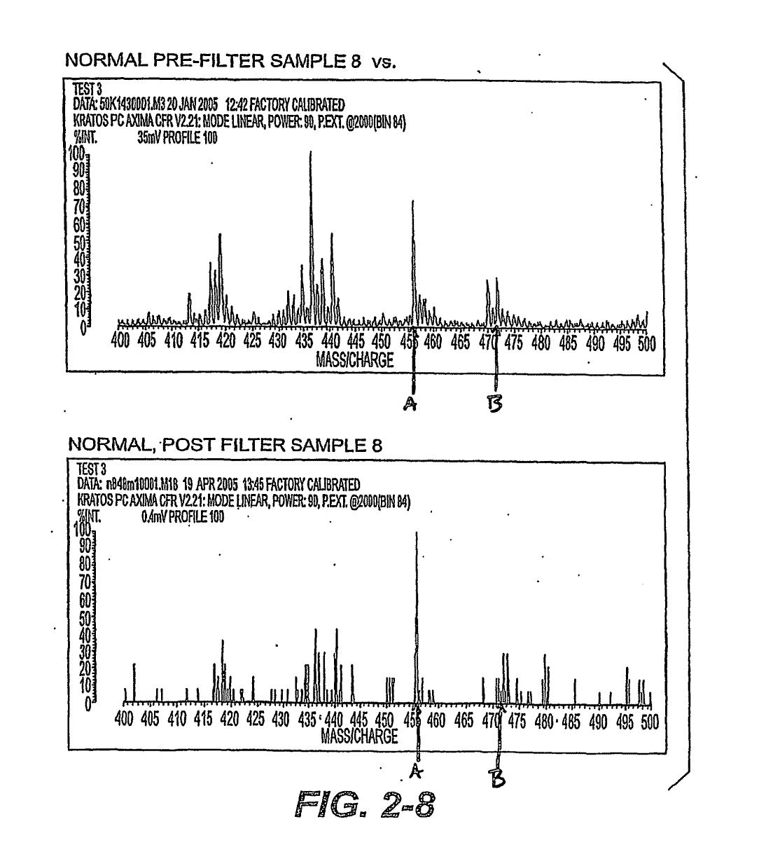

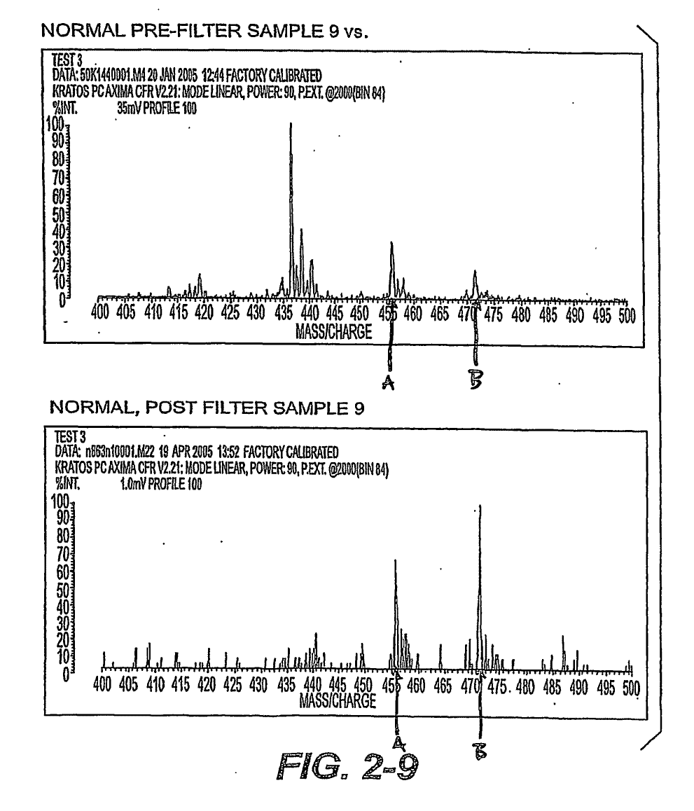

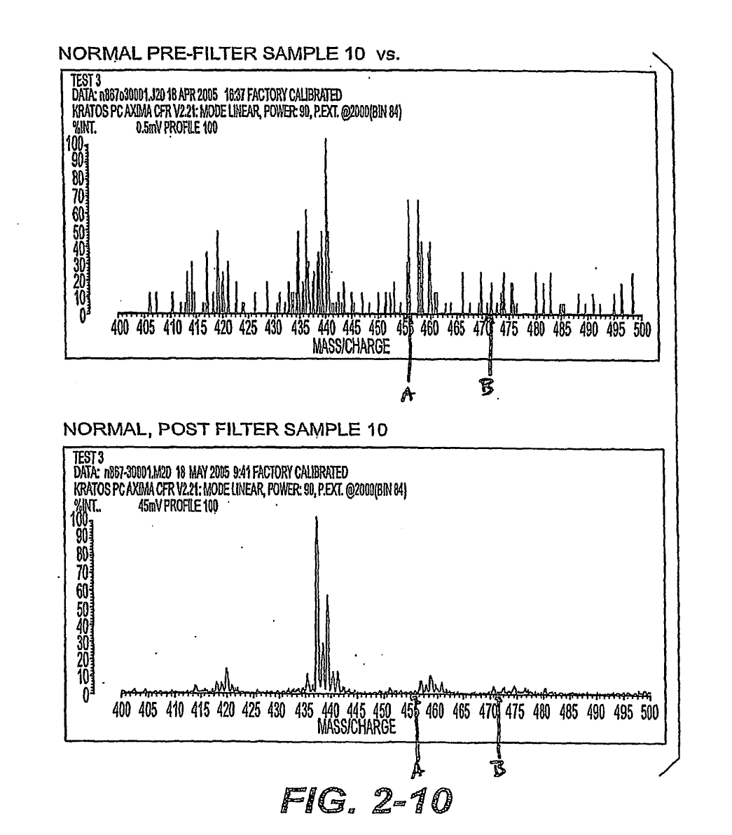

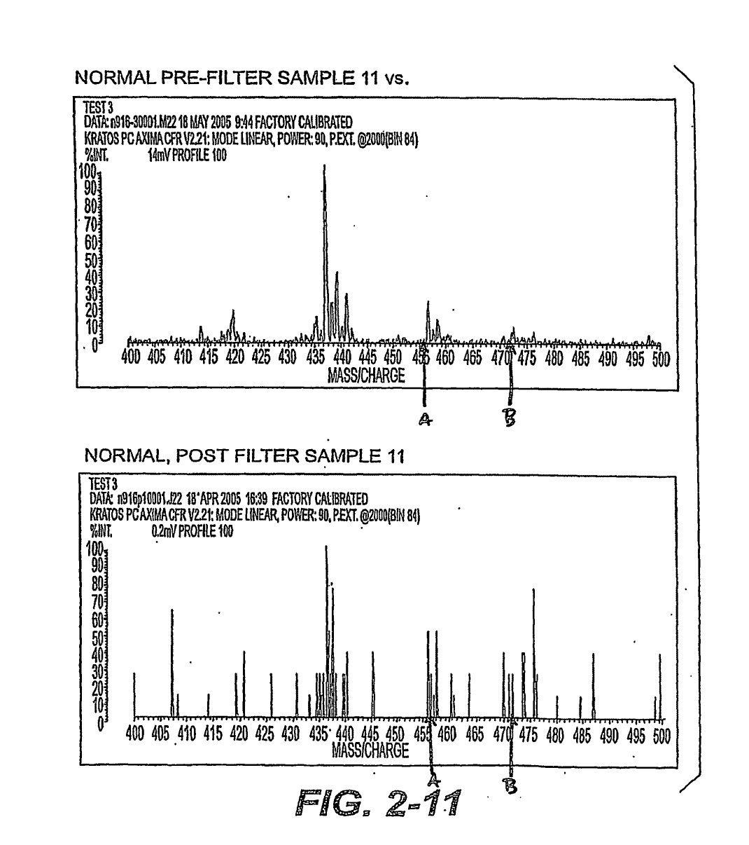

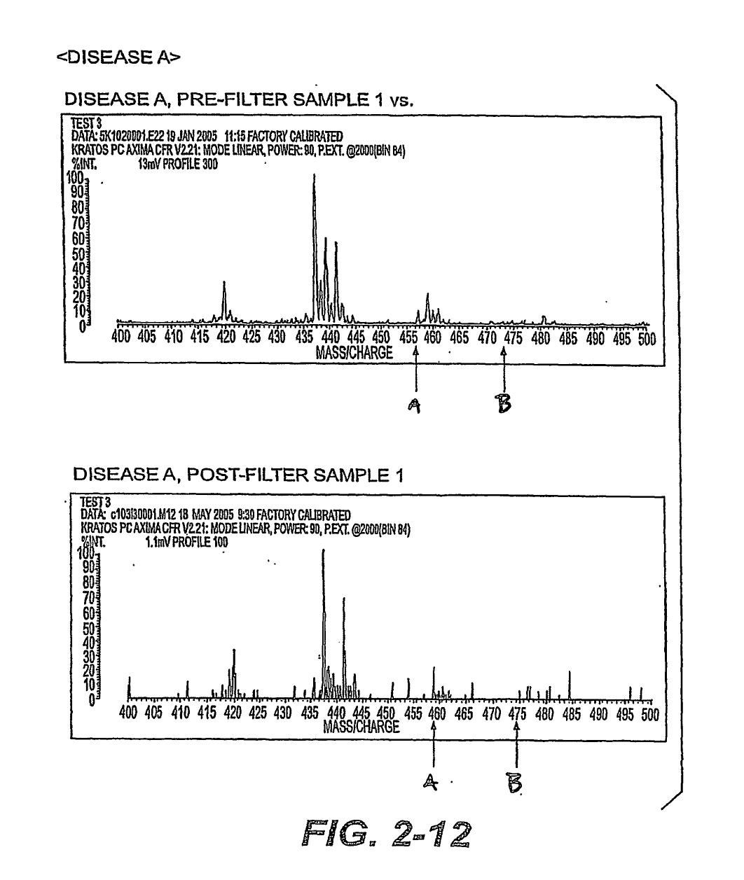

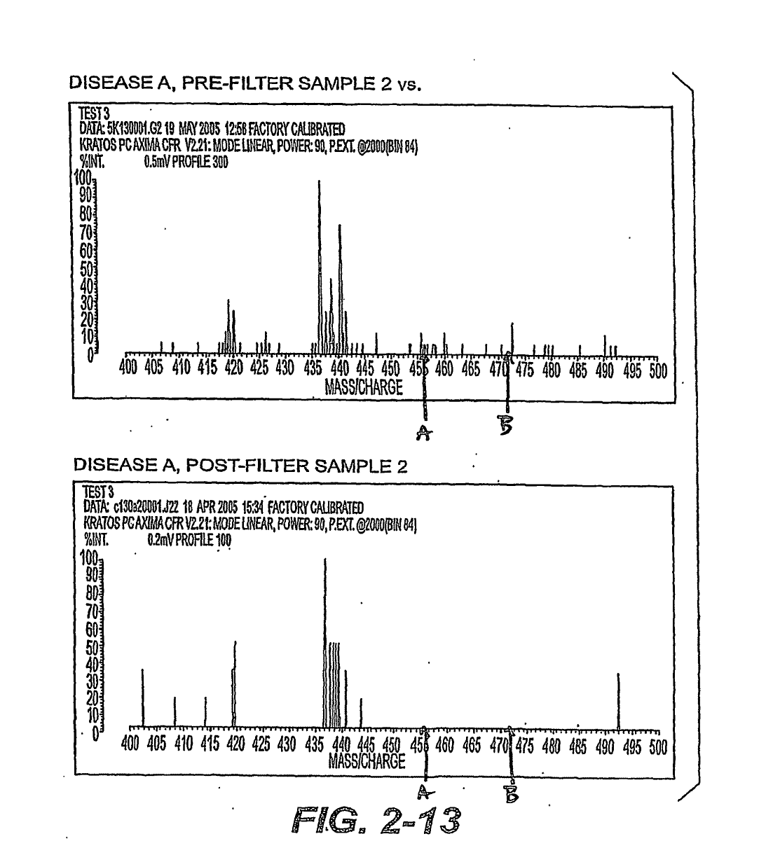

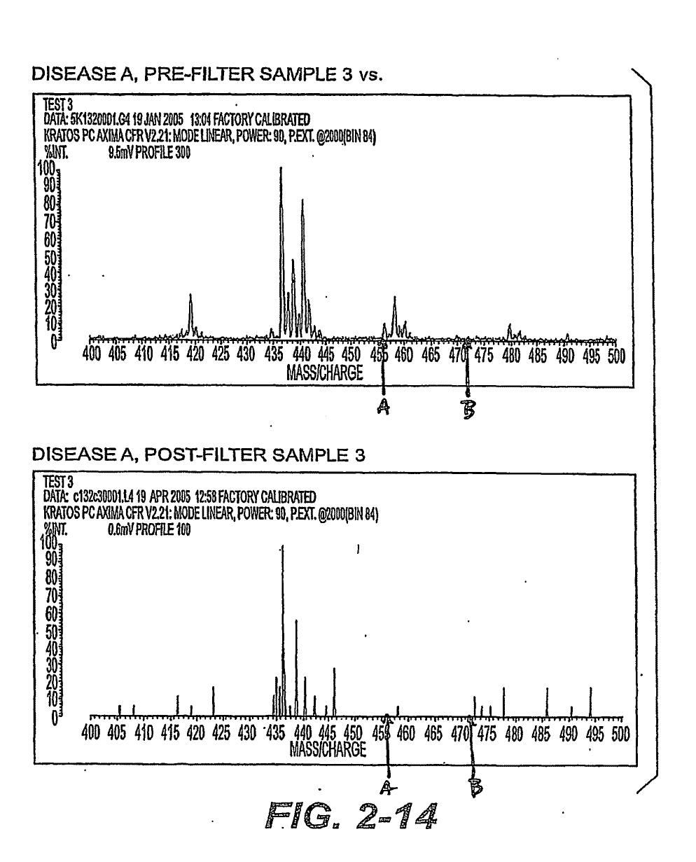

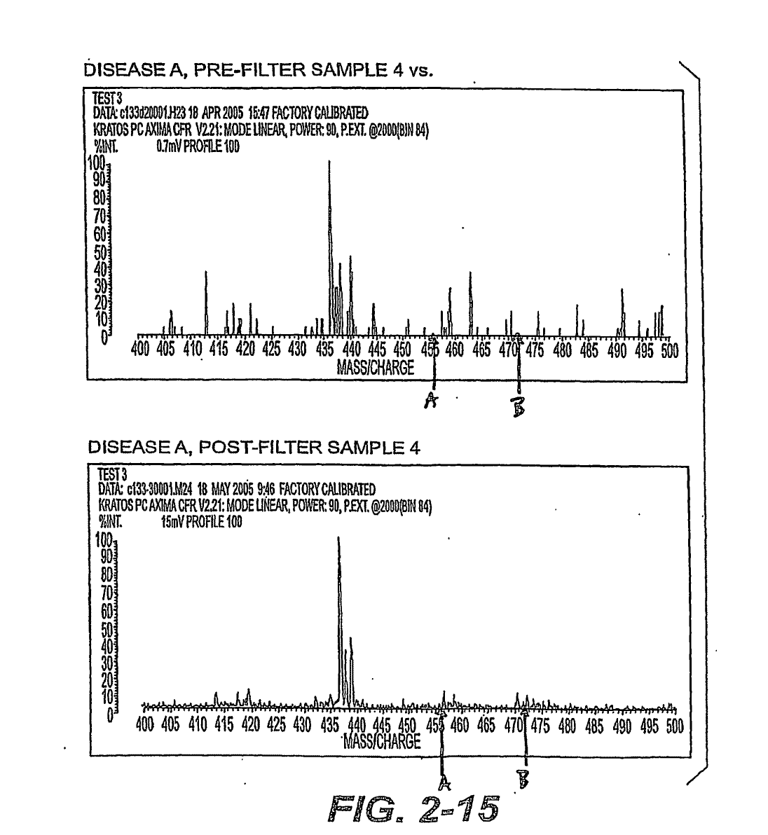

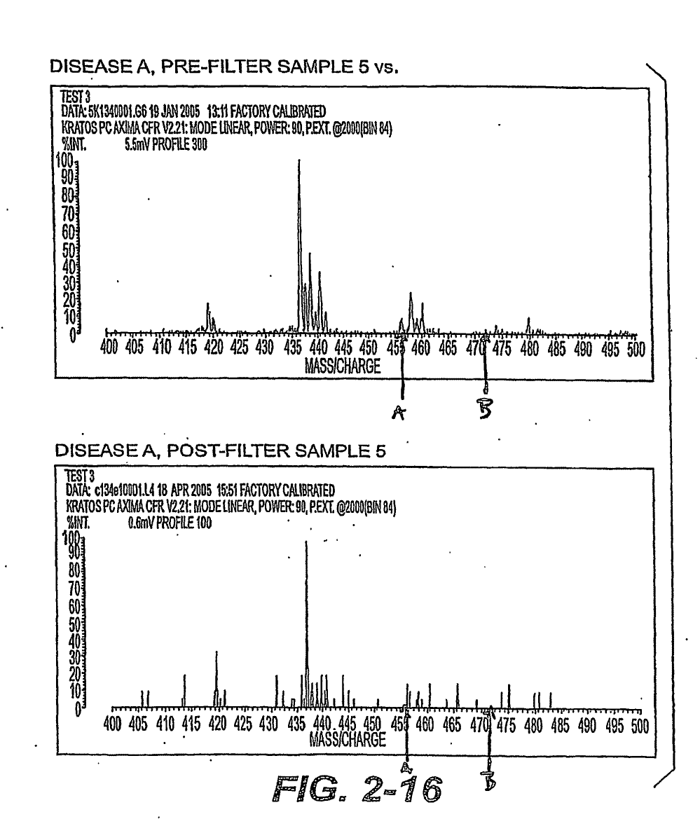

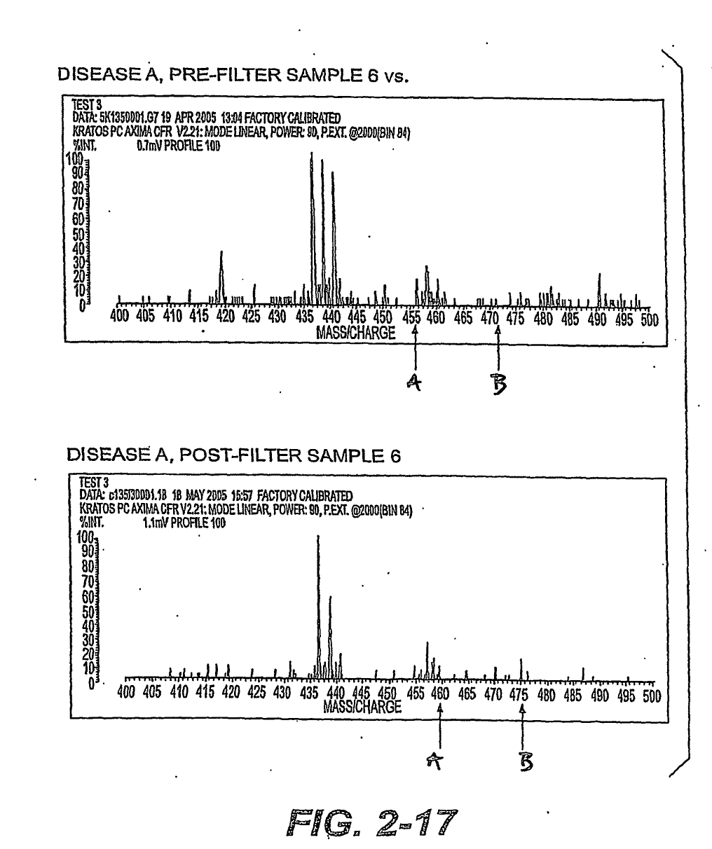

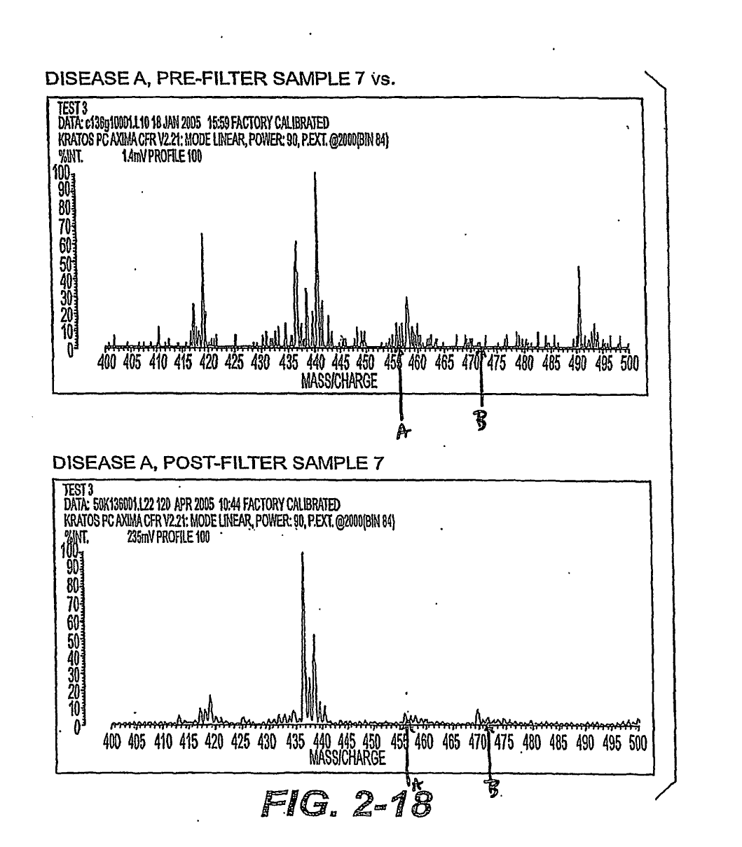

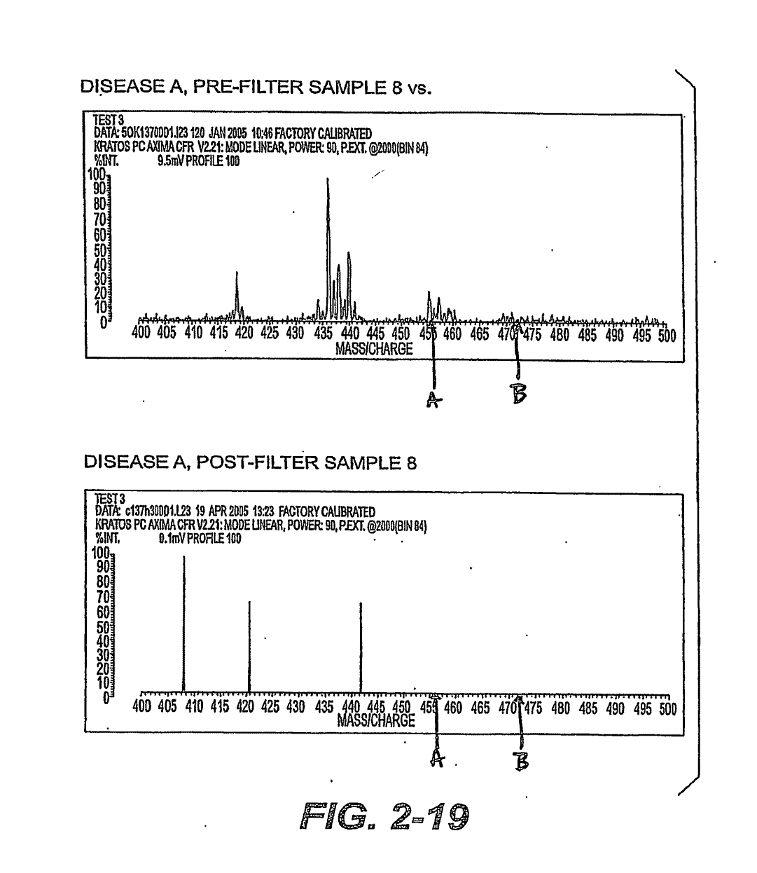

A comparison between normal sera data and lung cancer sera data is illustrated in FIGS. 2-1 to 2-19, with normal pre-filtered and post-filtered samples shown in FIGS. 2-1 to 2-11 and lung cancer (“Disease A”) pre-filtered and post-filtered samples shown in FIGS. 2-12 to 2-19. Further, FIGS. 6a-1 to 6a-32 show chromatograms of pre-filtered sera versus post-filtered sera of lung cancer patients and FIGS. 6b-1 to 6b-34 show the chromatograms of pre-filtered and post-filtered sera of normal patients.

First, normal sera and lung cancer sera were tested in the pre-filtered condition. Eleven normal sera and eight lung cancer sera were treated by the same method as described above and the chromatogram profiles shown in the figures were created. When the chromatogram profiles were compared on a fixed range, a substantial difference was found between normal sera and lung cancer sera in the spots corresponding to points A and B in FIGS. 2-1 to 2-19. As illustrated in the FIGS., point A corresponds to a mass over charge ratio of 456 and point B corresponds to a mass over charge ratio of 472. As seen in the figures, the chromatograms of normal sera showed peaks in the spots A and B. However, the chromatograms of lung cancer sera had substantially no peaks at spots A and B.

In the case of normal sera as shown in FIGS. 2-1 to 2-11, ten of eleven samples showed peaks at spot A. Six samples of eleven samples show peaks in the spot B. However, in the case of lung cancer sera, as shown in FIGS. 2-12 to 2-19, all eight samples show no peaks in the spots A and B. The table below summarizes these results:

Spot

| Sera Group | A | B |
|---|---|---|
| No. of samples with | 10/11 | 6/11 |
| peaks in Normal Sera | | |
| No. of samples with | 0/8 | 0/8 |
| peaks in Lung Cancer | | |

Next, normal sera data and lung cancer sera data were tested after filtering. The normal sera and lung cancer sera were treated by the same filtration method as described above. The chromatogram profiles were prepared from the filtered sera. When the chromatogram profiles were compared on a fixed range with normal sera, all eleven samples showed peaks in the spot A and seven of eleven samples showed peaks in the spot B. However, in case of lung cancer sera, the result was the same as the pre-filtered data. As illustrated in FIGS. 2-12 to 2-19, all eight samples show no peaks in the spot A and B. The table below summarizes these results:

Spot

| Sera Group | A | B |
|---|---|---|
| No. of samples with | 11/11 | 7/11 |
| peaks in Normal Sera | | |
| No. of samples with | 0/8 | 0/8 |
| peaks in Disease A Sera | | |

It should be noted, in the case of lung cancer sera, low peaks in the spots of A and B were reduced or eliminated after filtering the sera. In other words, the use of the filter accentuated the differences in the mass spectrographs of sera from people with lung cancer compared to sera from people not suffering from lung cancer. This enhanced the detection of the peaks.

The resulting data, as shown in FIGS. 2-1 to 2-19 and 6a-1 to 6b-34, show that the use of the filter accentuates the differences between the pre-filter chromatograms and the post-filter chromatograms. This enhancement improves the detection of the peaks.

In particular, the data presented in the above tables and the chromatograms illustrated in FIGS. 2-1 to 2-19 illustrate that filtering of the samples resulted in a ten percent increase in sensitivity at spot A and a nine percent increase in sensitivity at spot B in the normal chormatograms. As shown in the tables, in ten of the eleven normal chromatograms, peaks at spot A were shown pre-filtering while all of the normal chromatograms illustrated peaks at spot A after filtering. Likewise, in six of the eleven normal chromatograms, peaks at spot B were shown pre-filtering while seven of the normal chromatograms illustrated peaks at spot B after filtering.

Furthermore, the data presented in FIGS. indicates an increase in specificity in the chromatograms of samples having the disease. Six out of eight (FIGS. 2-12, 2-14, 2-16, 2-17, 2-18 and 2-19) of the pre-filtered lung cancer samples show a peak at spot A. However, post-filtering, these peaks are no longer present. Thus, a twenty-five percent increase in specificity was realized in the specificity of detection of spot A.

One of ordinary skill in the art will realize that, while the above example specifically discusses lung cancer, the present invention is not meant to be limited to any particular disease. The present invention is applicable to any disease that may show a difference in mass chromatograms compared to those of normal patients. Exemplary diseases may include, but are not limited to, cancers of the respiratory, gastrointestinal, renal, CNS, endocrine and blood systems or any other diseases or disease processes (e.g. necrosis, apoptosis) in which there are potential alterations in molecules contained in biological fluid (e.g. blood and blood derivatives, urine, cerebral spinal fluid, sputum, lavage). Such biological molecules may include, but are not limited to, macromolecules such as polypeptides, proteins, nucleic acids, enzymes, DNA, RNA, polynucleotides, oligonucleotides, carbohydrates, oligosaccharides, polysaccharides, fragments of biological macromolecules (e.g. nucleic acid fragments, peptide fragments, and protein fragments), complexes of biological macromolecules (e.g. nucleic acid complexes, protein-DNA complexes, receptor-ligand complexes, enzyme-substrate, enzyme inhibitors, peptide complexes, protein complexes, carbohydrate complexes, and polysaccharide complexes), small biological molecules such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphospho-sugars, synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents; and synthetic polymers, oligomers, and copolymers. Additionally, any suitable mixture or combination of the substances mentioned above may also be included in the biological samples.

### EXAMPLE 2

The discussion below discusses a specific pancreatic cancer screening performed using the apparatus and methods according to the present invention discussed above. MALDI-TOF-MS was used to generate a spectra sample data set representing distinct protein/peptide patterns in serum. In one clinical investigation, fluid either from patients with pancreatic cancer or healthy controls were obtained before surgical procedures. All final diagnoses were confirmed by histopathology and all controls were at high risk for pancreatic cancer, but without evidence of pancreatic cancer based on clinical presentation and CT scan examination.

The sera were prepared for evaluation by the mass spectrometer by making a matrix of serum samples. The mass spectrometer matrix contained saturated alpha-cyano-4-hydroxycinnamic acid in 50% acetonitrile-0.05% trifluoroacetic acid (TFA). The fluids were diluted 1:1000 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear, mass range, 0 to about 5,000; laser power, 90; profile, 100; shots per spot, 5. The output of the mass spectrometer was stored in computer storage in the form of a sample data set.

Before applying the filter, the serum was diluted 1:10 in 0.1% n-Octyl β-D-Glucopyranoside. The micro tubes were cut individually from Micro Amp 8 strip tubes. A hole was made in the bottom part of the micro tube by using a needle (Becton Dickinson 20G1) having a diameter of 0.9 mm or less. The micro tube with hole was placed on the metal plate of a gel-pak suction apparatus (air pump) and the hole was adjusted to the same air-flow direction of the air pump. The filter was placed on the top of the micro tube. 20 μl of serum was loaded on the upper part of filter. The serum solution spreads out, filling the inner square of filter. The negative air flow was applied by pumping the air pump manually. The dropped sera solutions from the filter to the micro tube were collected and transferred to the new tube. The filtered serum was further diluted 1:100 in 0.1% n-Octyl β-D-Glucopyranoside. 0.5 μL, of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

As with the lung cancer screening discussed above with respect to Example 1, the use of the filter accentuated the differences between the presence of peaks in the pre-filter and post-filter chromatograms for pancreatic cancer. These results will be apparent to one of ordinary skill in the art by examining FIGS. 7-1 to 7-42 in the same manner as the examination of the results of the lung cancer screening discussed above. Clearly, the use of the filter enhanced the detection of the relevant peaks.

One of ordinary skill in the art will realize that, while the above example specifically discusses pancreatic cancer, the present invention is not meant to be limited to any particular disease. The present invention is applicable to any disease that may show a difference in mass chromatograms compared to those of normal patients. Exemplary diseases may include, but are not limited to, cancers of the respiratory, gastrointestinal, renal, CNS, endocrine and blood systems or any other diseases or disease processes (e.g. necrosis, apoptosis) in which there are potential alterations in molecules contained in biological fluid (e.g. blood and blood derivatives, urine, cerebral spinal fluid, sputum, lavage). Such biological molecules may include, but are not limited to, macromolecules such as polypeptides, proteins, nucleic acids, enzymes, DNA, RNA, polynucleotides, oligonucleotides, carbohydrates, oligosaccharides, polysaccharides, fragments of biological macromolecules (e.g. nucleic acid fragments, peptide fragments, and protein fragments), complexes of biological macromolecules (e.g. nucleic acid complexes, protein-DNA complexes, receptor-ligand complexes, enzyme-substrate, enzyme inhibitors, peptide complexes, protein complexes, carbohydrate complexes, and polysaccharide complexes), small biological molecules such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphospho-sugars, synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents; and synthetic polymers, oligomers, and copolymers. Additionally, any suitable mixture or combination of the substances mentioned above may also be included in the biological samples.

### EXAMPLE 3

The discussion below discusses a specific bladder cancer screening performed using the apparatus and methods according to the present invention discussed above. MALDI-TOF-MS was used to generate a spectra sample data set representing distinct protein/peptide patterns in urine. In one clinical investigation, urine either from patients with bladder cancer or healthy controls were obtained before surgical procedures. All final diagnoses were confirmed by histopathology and all controls were at high risk for bladder cancer, but without evidence of bladder cancer based on clinical presentation and CT scan examination.

The samples were prepared for evaluation by the mass spectrometer by making a matrix of urine samples. The mass spectrometer matrix contained saturated alpha-cyano-4-hydroxycinnamic acid in 50% acetonitrile-0.05% trifluoroacetic acid (TFA). The fluids were diluted 1:1000 in 0.1% n-Octyl β-D-Glucopyranoside. 0.5 μL of the matrix was placed on each defined area of a sample plate with 384 defined areas and 0.5 μL urine from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Before applying the filter, the urine was diluted 1:10 in 0.1% n-Octyl β-D-Glucopyranoside. The micro tubes were cut individually from Micro Amp 8 strip tubes. A hole was made in the bottom part of the micro tube by using needle (Becton Dickinson 20G1) having a diameter of 0.9 mm or less. The micro tube with hole was placed on the metal plate of a gel-pak suction apparatus (air pump) and the hole was adjusted to the same air-flow direction of the air pump. The filter was placed on the top of the micro tube. 20 μl of urine was loaded on the upper part of filter. The urine solution spreads out, filling the inner square of filter. The negative air flow was applied by pumping the air pump manually. The dropped urine solutions from the filter to the micro tube were collected and to transferred to the new tube. The filtered urine was further diluted 1:100 in 0.1% n-Octyl β-D-Glucopyranoside. 0.5 μL of the matrix was placed on each defined area of a sample plate with 384 defined areas and 0.5 μL urine from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

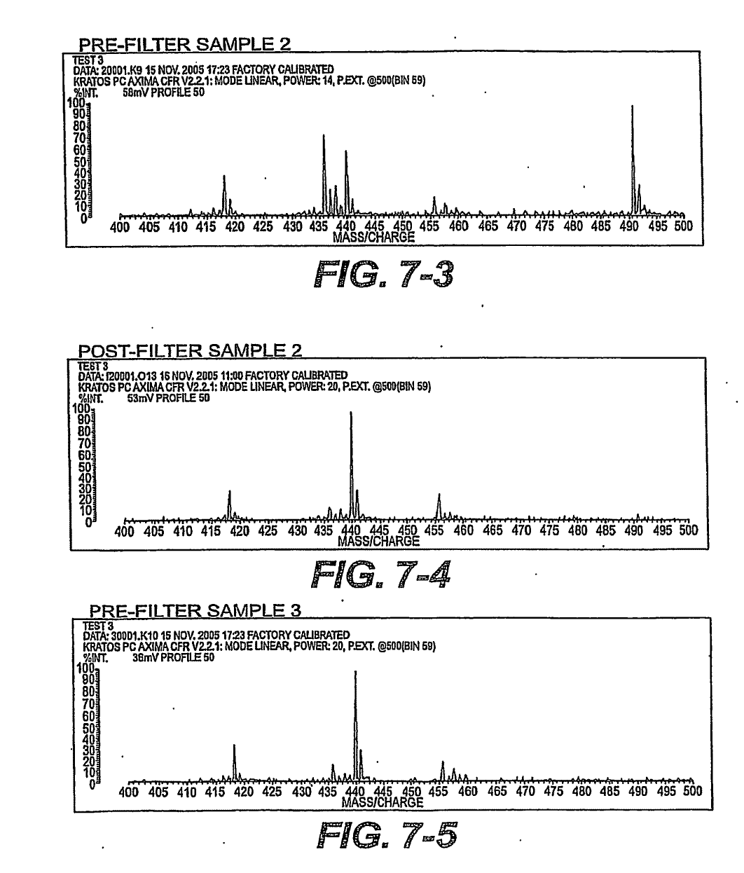

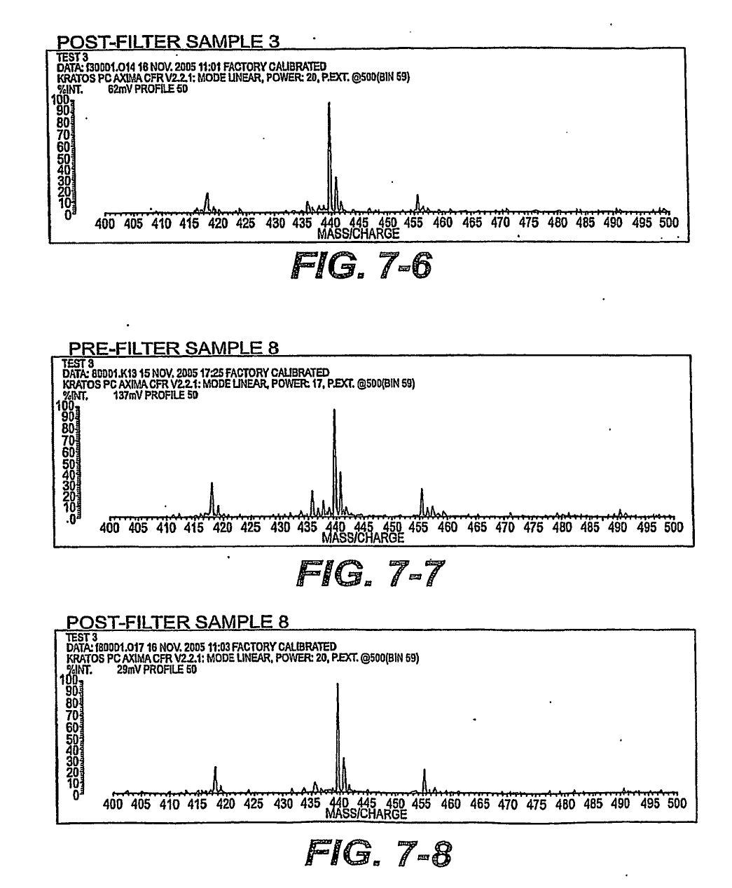

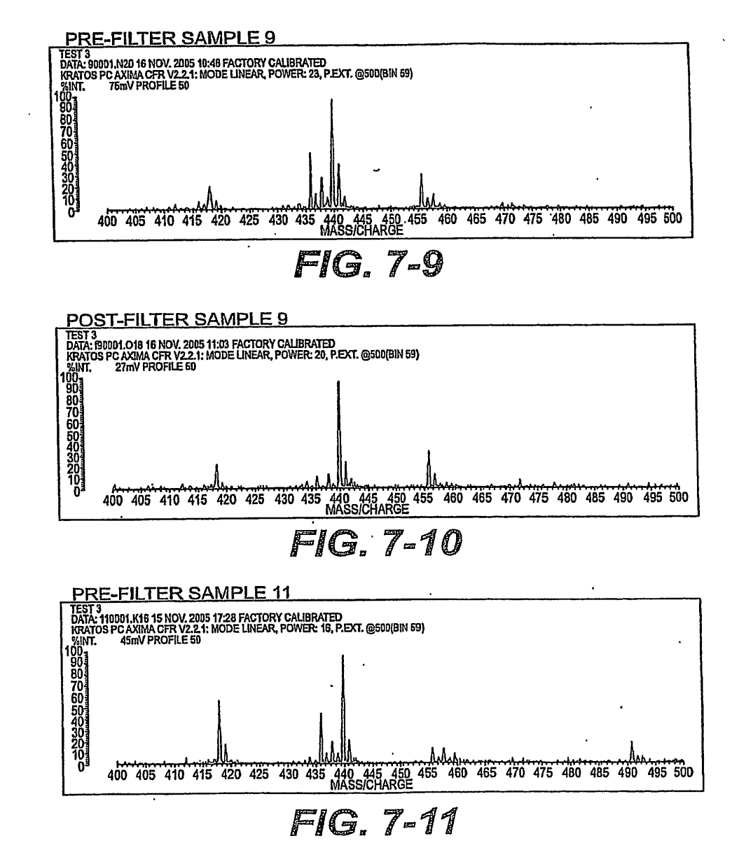

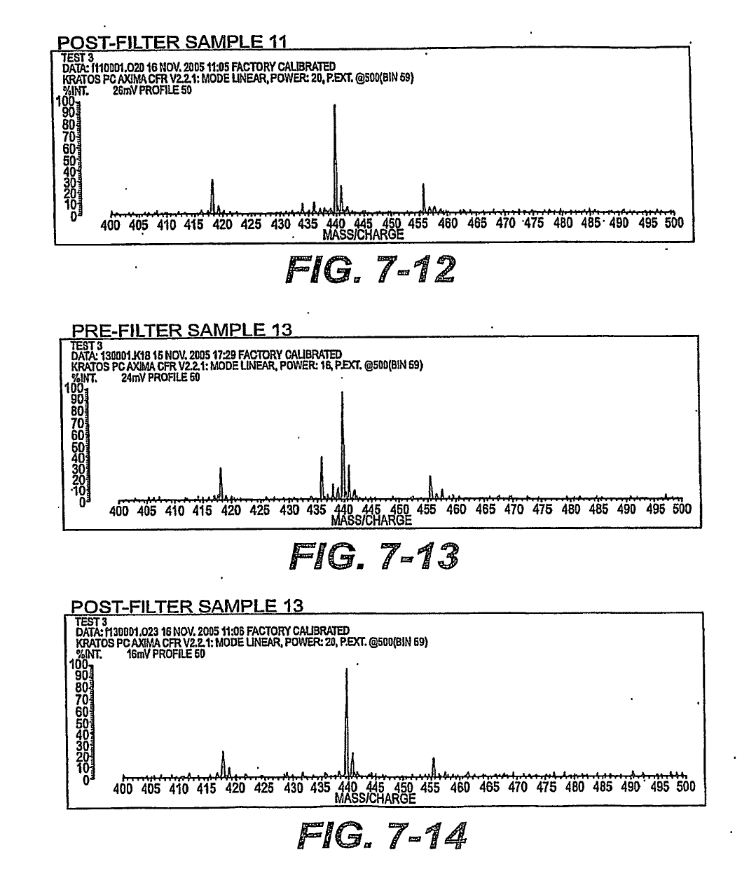

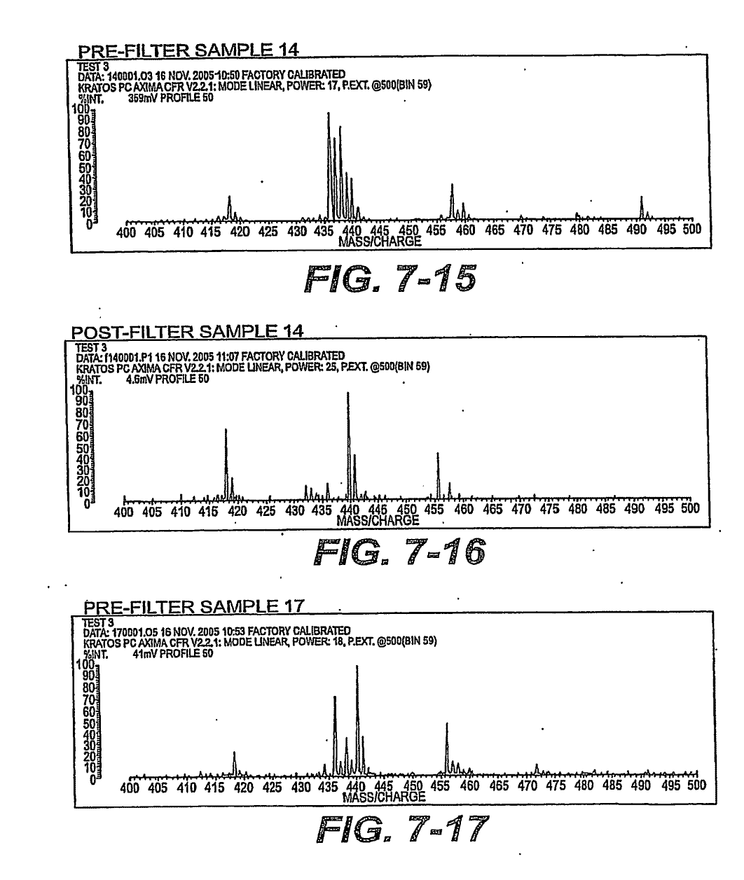

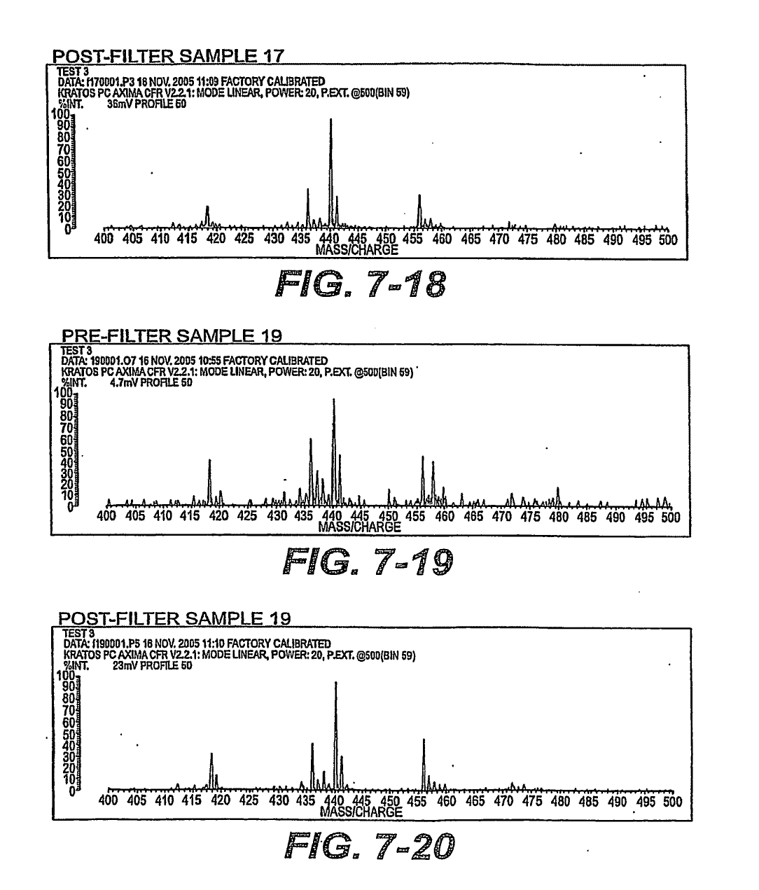

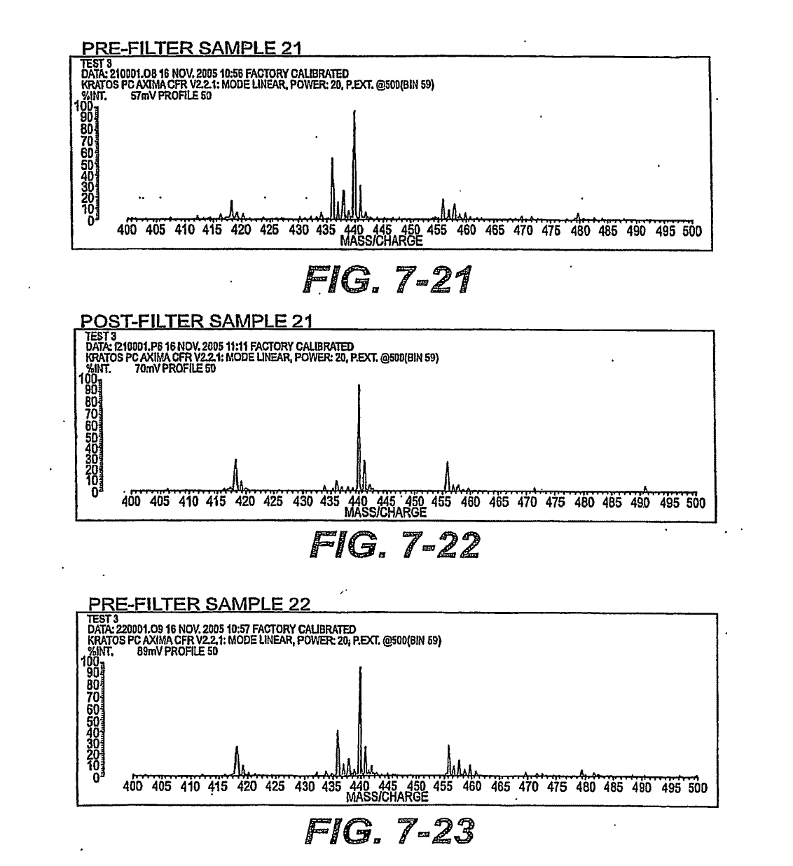

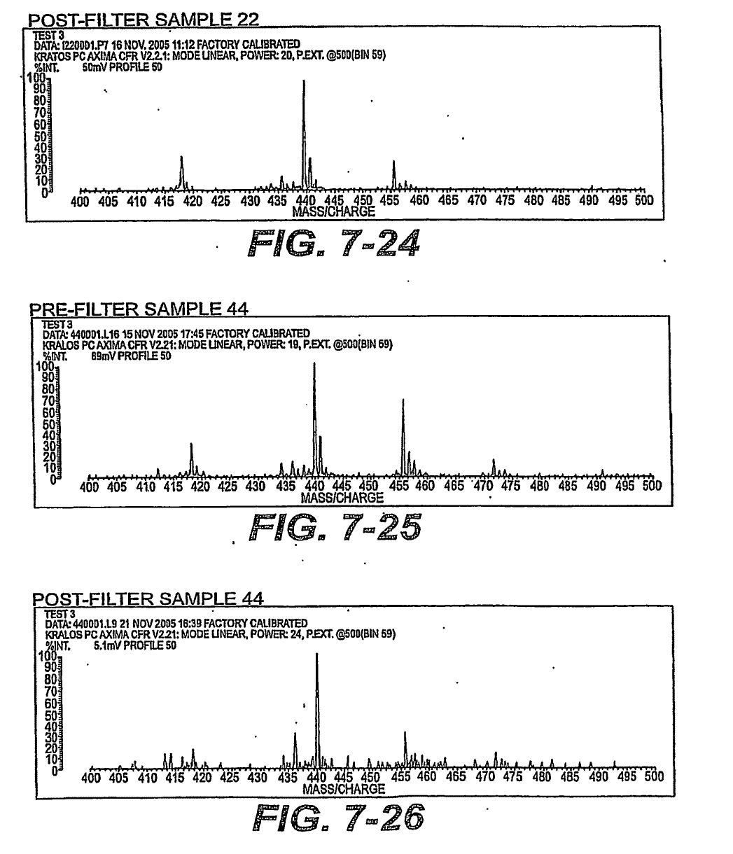

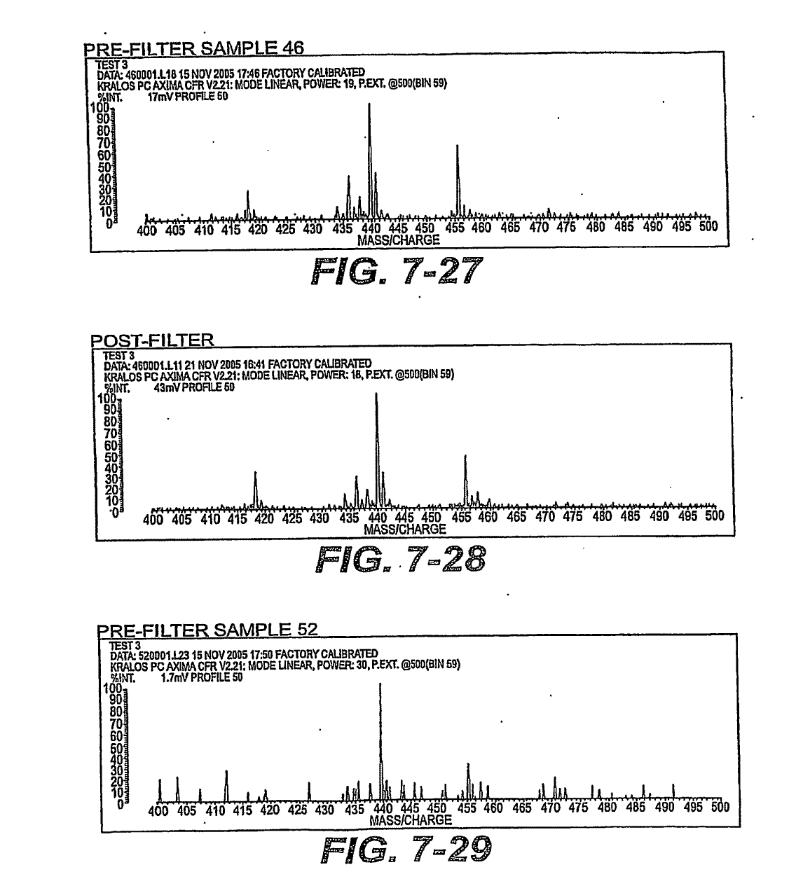

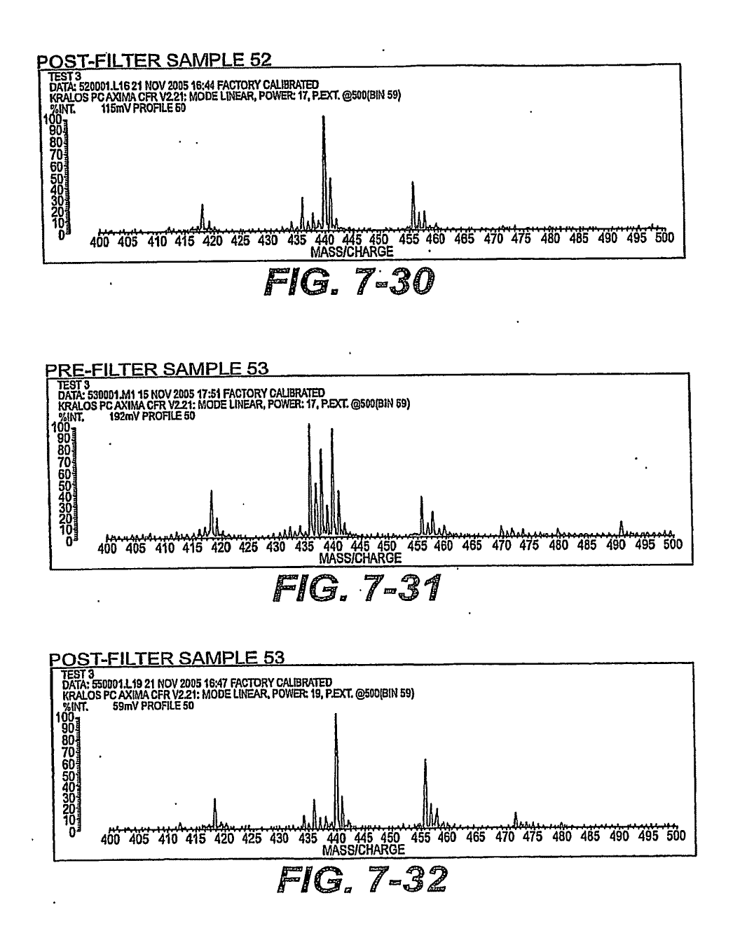

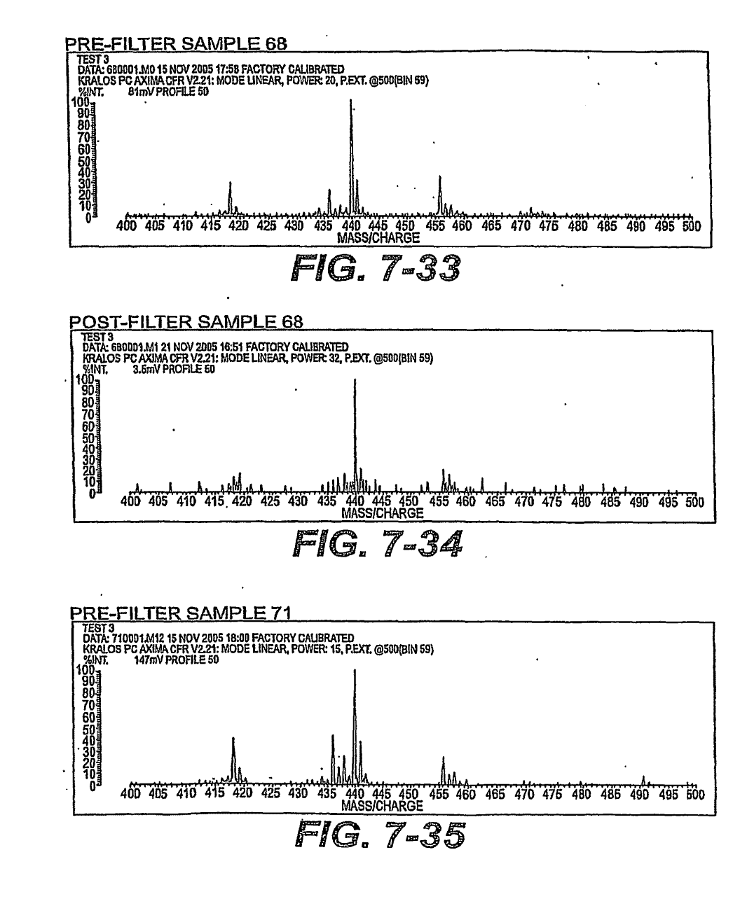

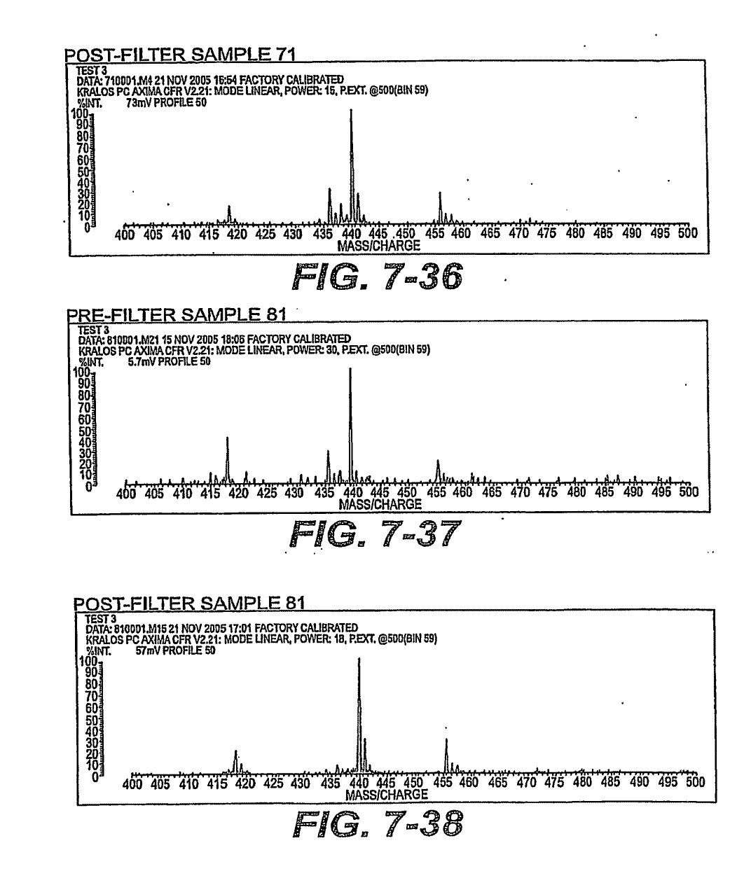

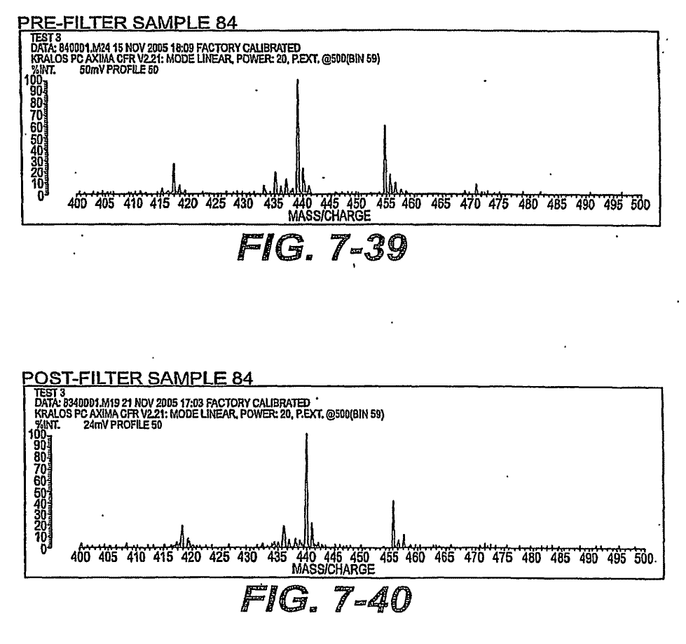

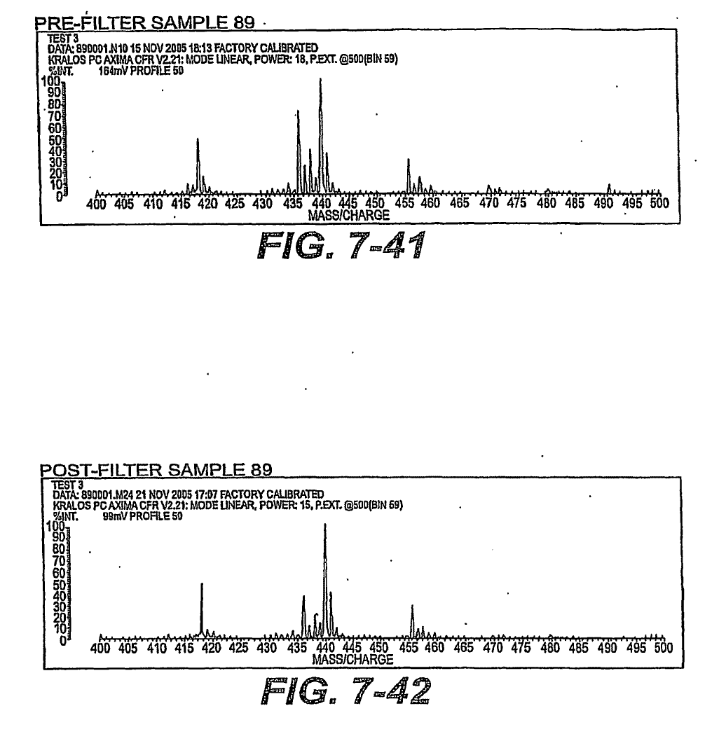

As with the lung cancer screening discussed above with respect to Example 1, the use of the filter accentuated the differences between the presence of peaks in the pre-filter and post-filter chromatograms for bladder cancer. These results will be apparent to one of ordinary skill in the art by examining FIGS. 8-1 to 8-12 (FIGS. 8-1 to 8-6 are chromatograms of samples known to have bladder cancer while FIGS. 8-7 to 8-12 are normal chromatograms). Clearly, the use of the filter enhanced the detection of the relevant peaks.

One of ordinary skill in the art will realize that, while the above example specifically discusses bladder cancer, the present invention is not meant to be limited to any particular disease. The present invention is applicable to any disease that may show a difference in mass chromatograms compared to those of normal patients. Exemplary diseases may include, but are not limited to, cancers of the respiratory, gastrointestinal, renal, CNS, endocrine and blood systems or any other diseases or disease processes (e.g. necrosis, apoptosis) in which there are potential alterations in molecules contained in biological fluid (e.g. blood and blood derivatives, urine, cerebral spinal fluid, sputum, lavage). Such biological molecules may include, but are not limited to, macromolecules such as polypeptides, proteins, nucleic acids, enzymes, DNA, RNA, polynucleotides, oligonucleotides, carbohydrates, oligosaccharides, polysaccharides, fragments of biological macromolecules (e.g. nucleic acid fragments, peptide fragments, and protein fragments), complexes of biological macromolecules (e.g. nucleic acid complexes, protein-DNA complexes, receptor-ligand complexes, enzyme-substrate, enzyme inhibitors, peptide complexes, protein complexes, carbohydrate complexes, and polysaccharide complexes), small biological molecules such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphospho-sugars, synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents; and synthetic polymers, oligomers, and copolymers. Additionally, any suitable mixture or combination of the substances mentioned above may also be included in the biological samples.

### EXAMPLE 4

The discussion below discusses a specific test used to illustrate the use of the method according to the various embodiments of the present invention for predicting response, or lack of response, of a patient to a particular disease treatment. MALDI-TOF-MS was used to generate a spectra sample data set representing distinct mass over charge ion peaks in serum. These peaks may represent biological molecules include macromolecules such as polypeptides, proteins, nucleic acids, enzymes, DNA, RNA, polynucleotides, oligonucleotides, carbohydrates, oligosaccharides, polysaccharides, fragments of biological macromolecules (e.g. nucleic acid fragments, peptide fragments, and protein fragments), complexes of biological macromolecules (e.g. nucleic acid complexes, protein-DNA complexes, receptor-ligand complexes, enzyme-substrate, enzyme inhibitors, peptide complexes, protein complexes, carbohydrate complexes, and polysaccharide complexes), small biological molecules such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphospho-sugars, synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents; and synthetic polymers, oligomers, and copolymers. Additionally, any suitable mixture or combination of the substances mentioned above may also be included in the biological samples.

The sera were prepared for evaluation by the mass spectrometer by making a matrix of serum samples. The mass spectrometer matrix contained saturated alpha-cyano-4-hydroxycinnamic acid in 50% acetonitrile-0.05% tri nuoroacetic acid (TFA). The sera were diluted 1:1000 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Before applying the filter, the serum was diluted 1:10 in 0.1% n-Octyl β-D-Glucopyranoside. The micro tubes were cut individually from Micro Amp 8 strip tubes. A hole was made in the bottom part of the micro tube by using a needle (Becton Dickinson 20G1) having a diameter of 0.9 mm or less. The micro tube with hole was placed on the metal plate of a gel-pak suction apparatus (air pump) and the hole was adjusted to the same air-flow direction of the air pump. The filter was placed on the top of the micro tube. 20 μl of serum was loaded on the upper part of filter. The serum solution spread out, filling the inner square of filter. The negative air flow was applied by pumping the air pump manually. The dropped sera solutions from the filter to the micro tube were collected and to transferred to the new tube. The filtered serum was further diluted 1:100 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

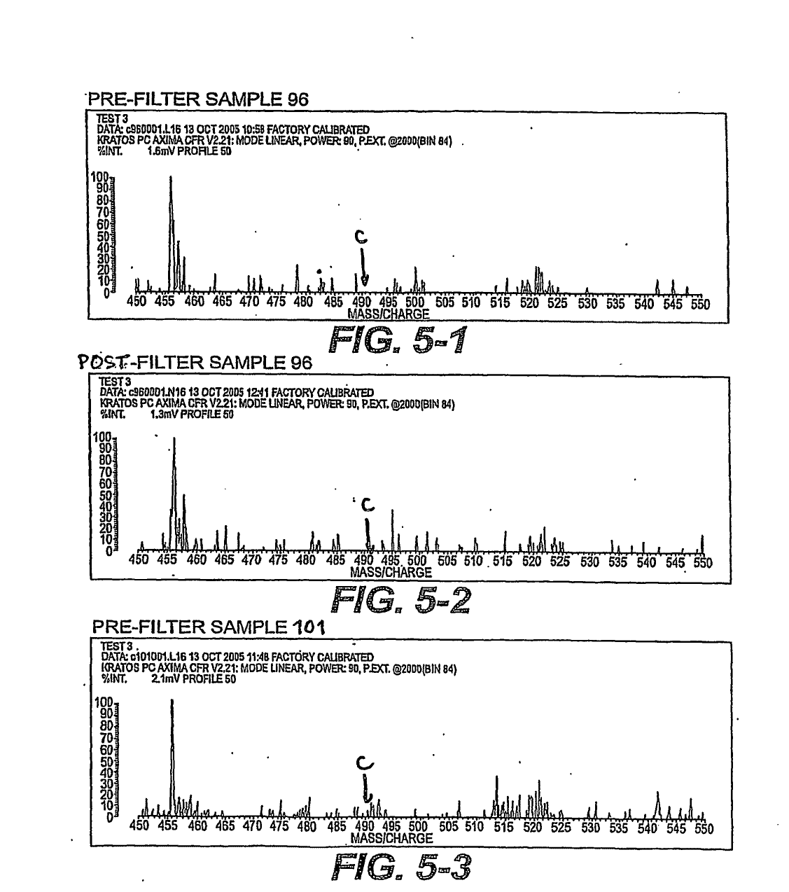

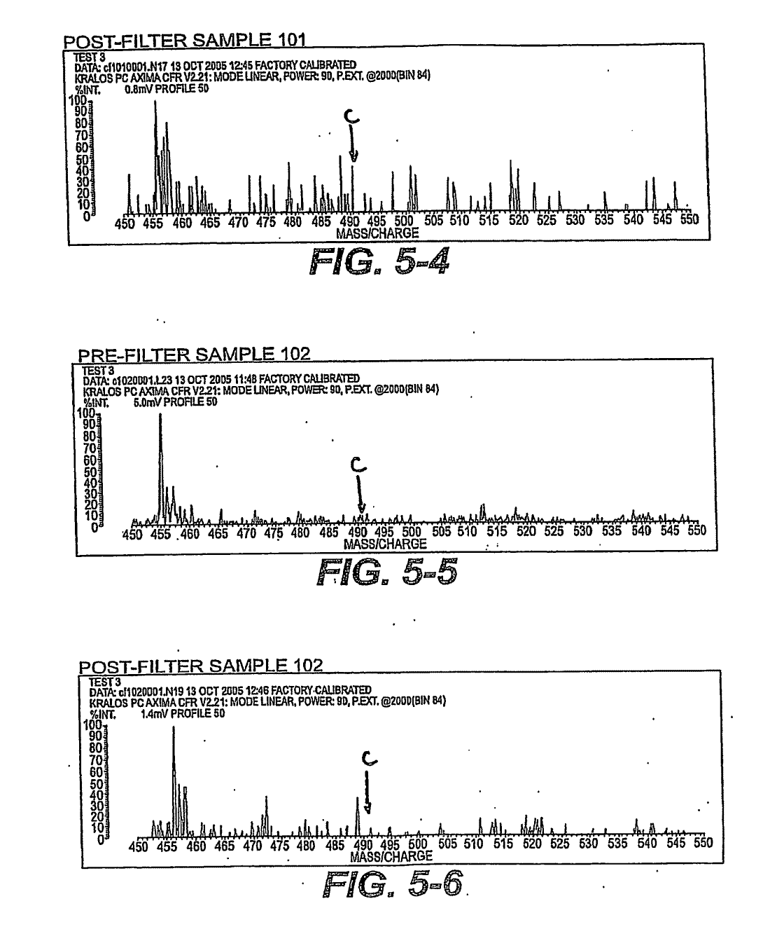

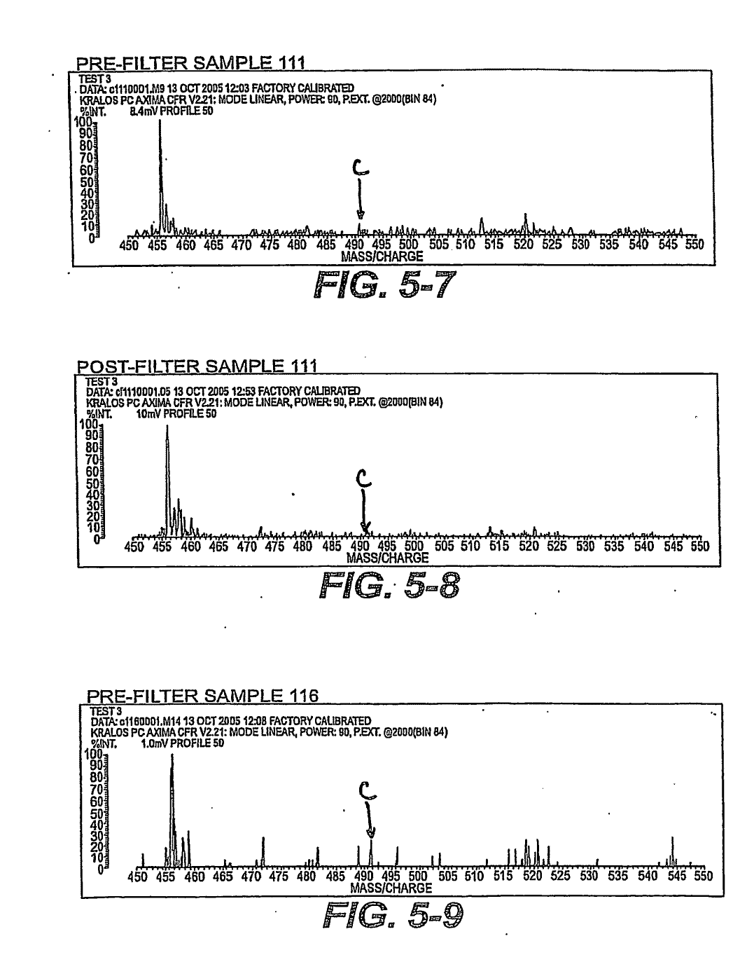

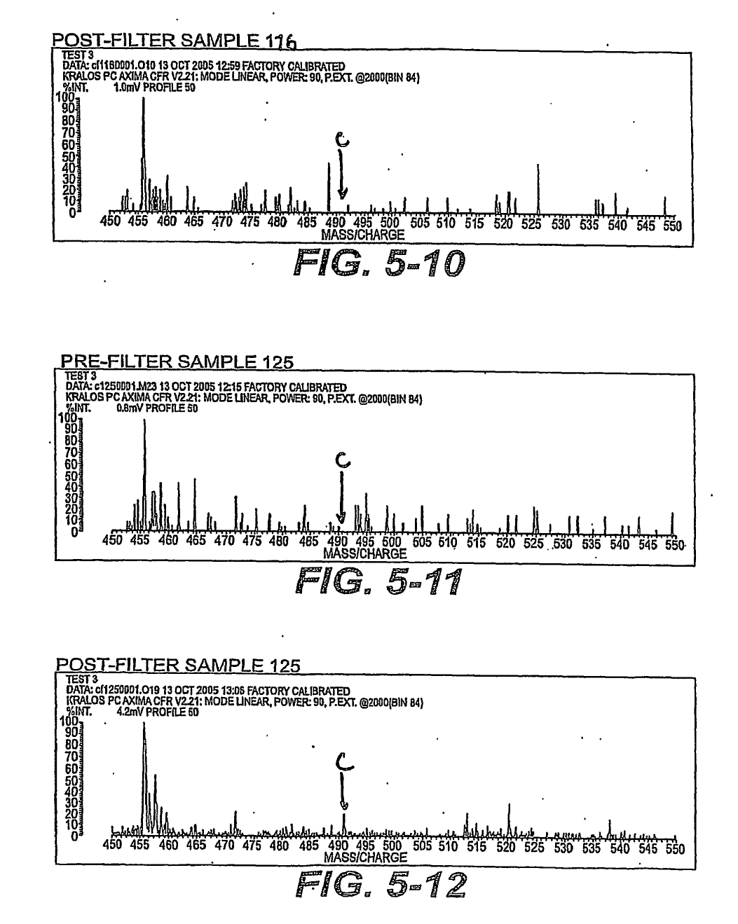

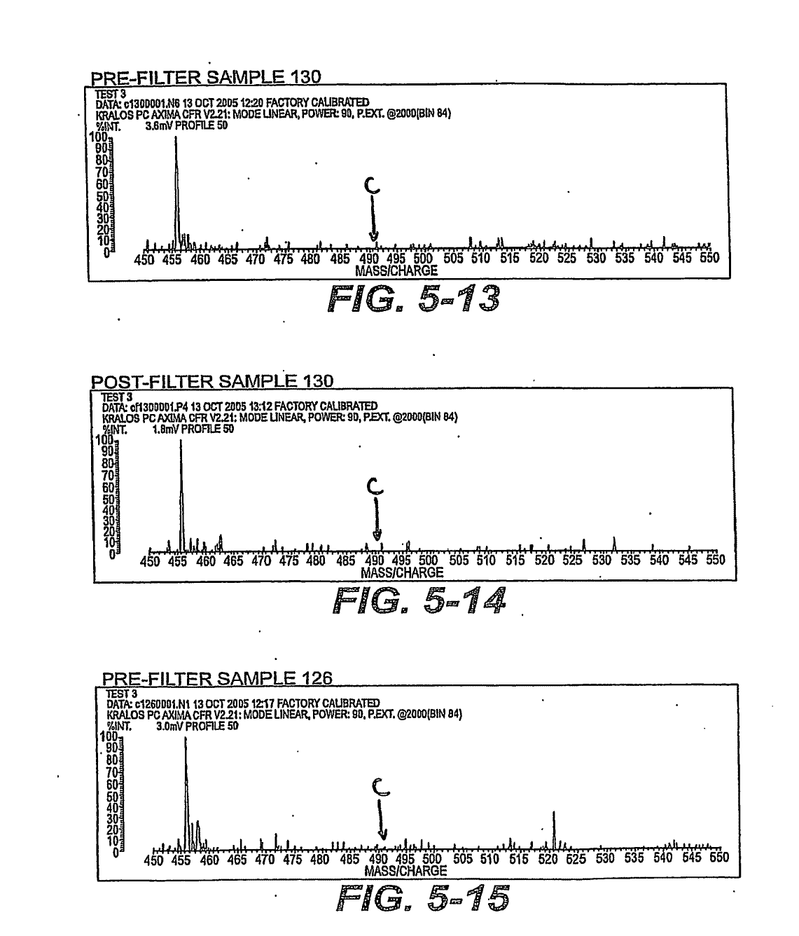

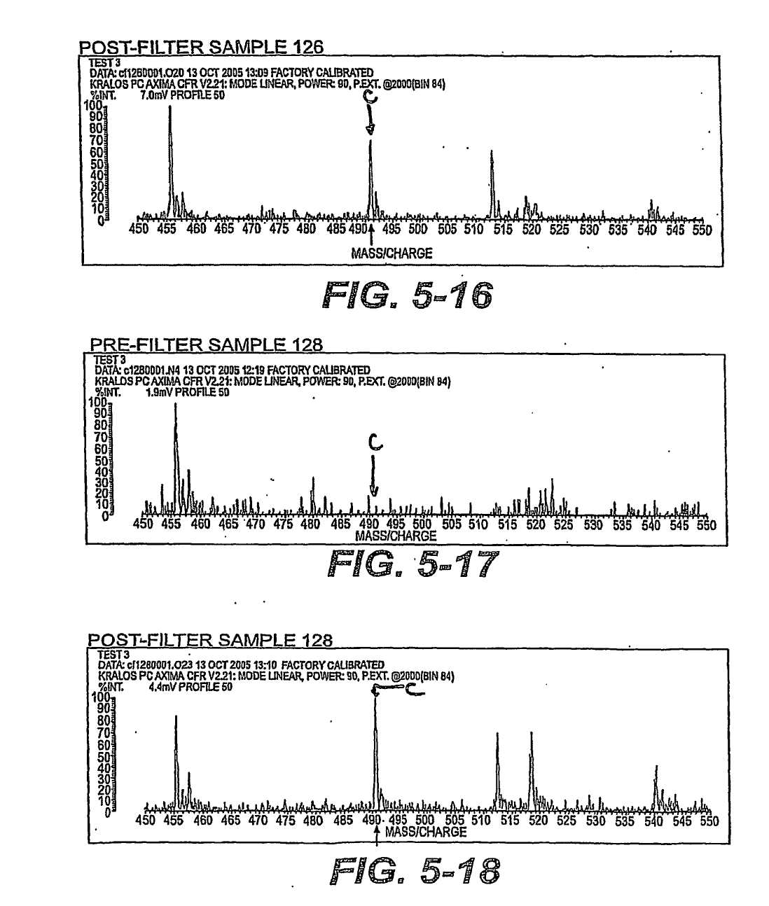

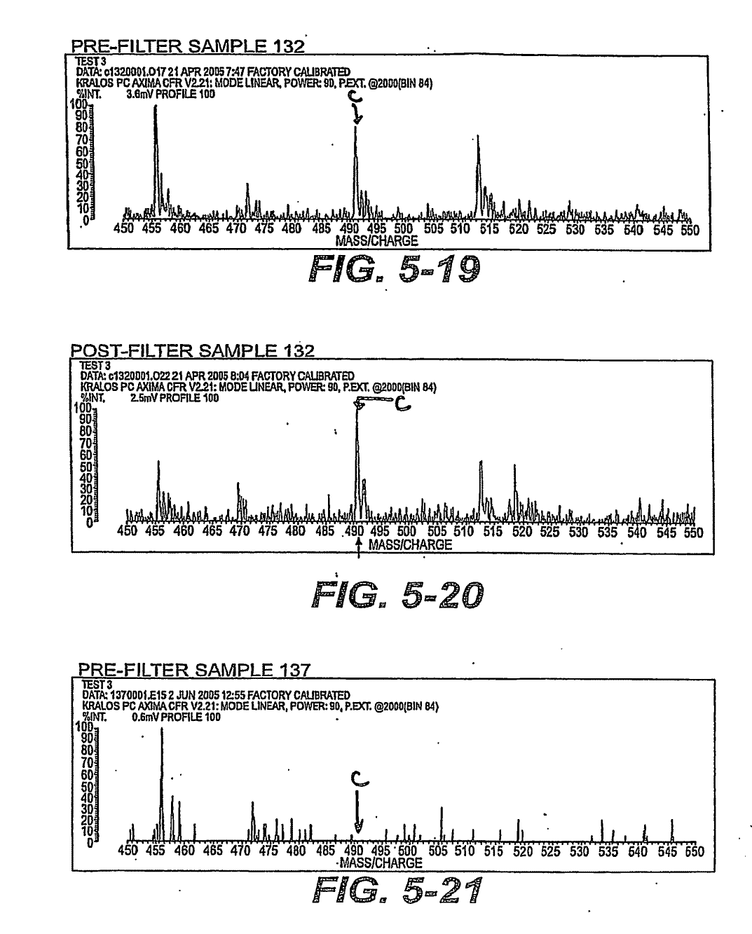

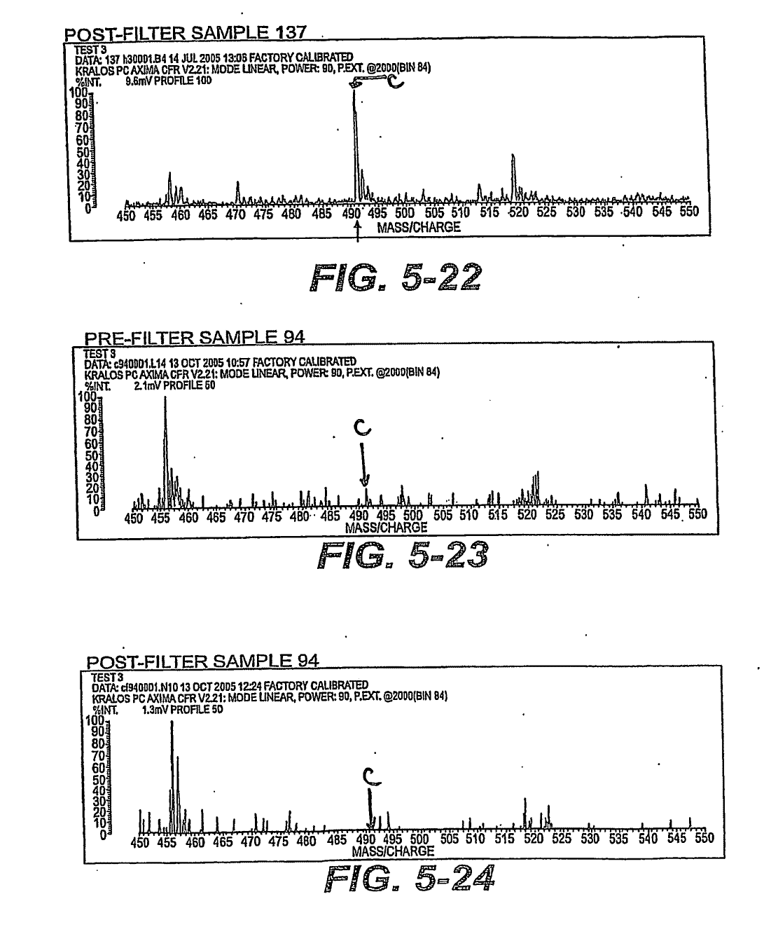

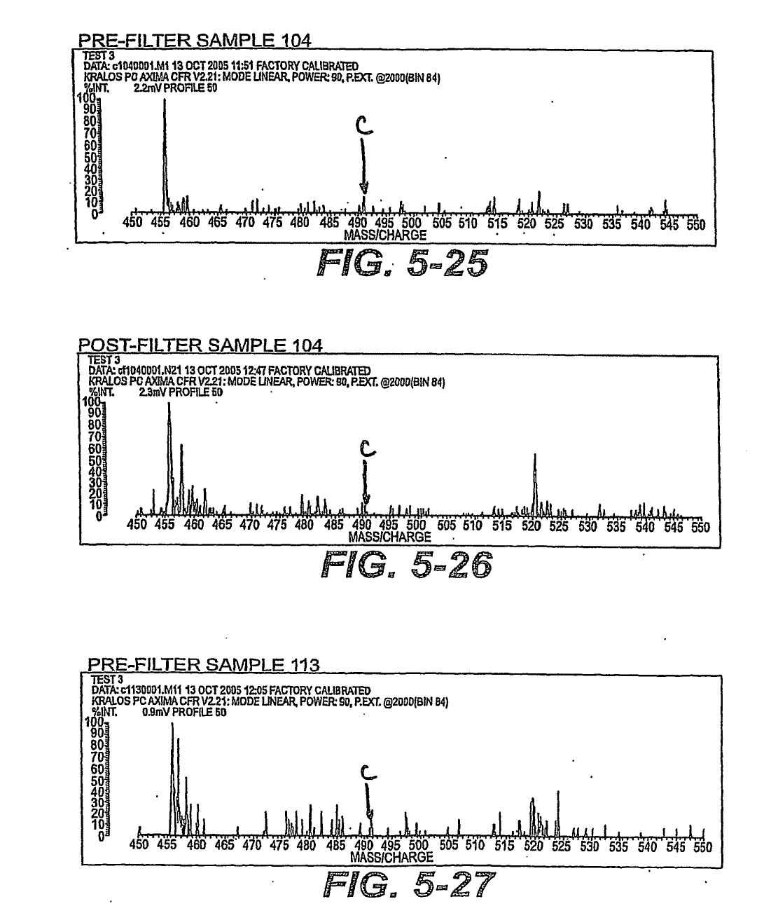

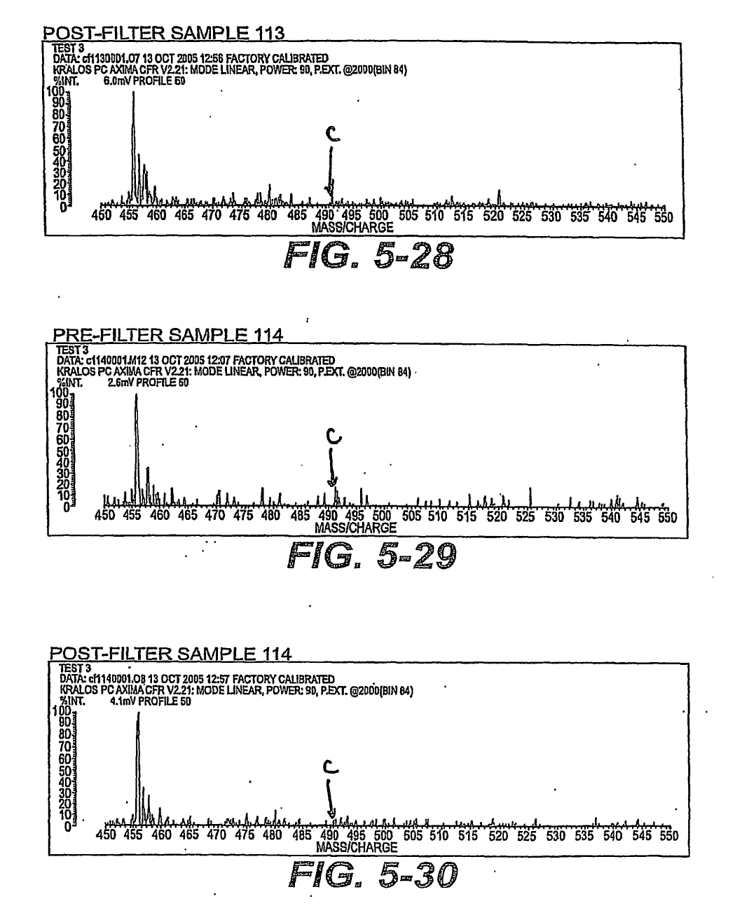

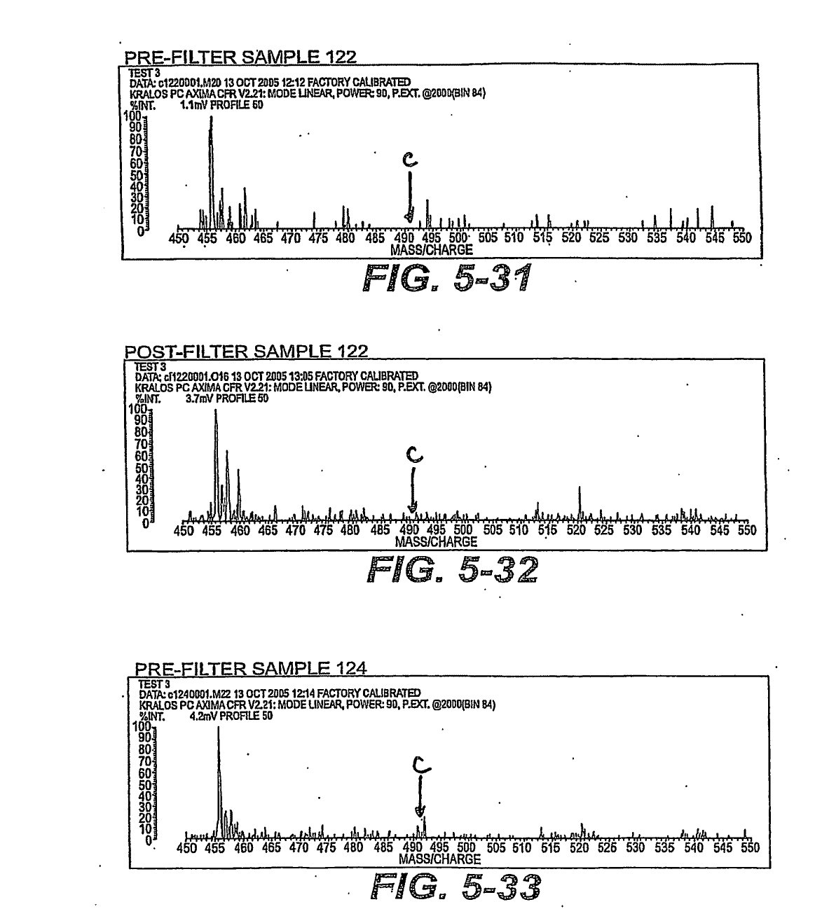

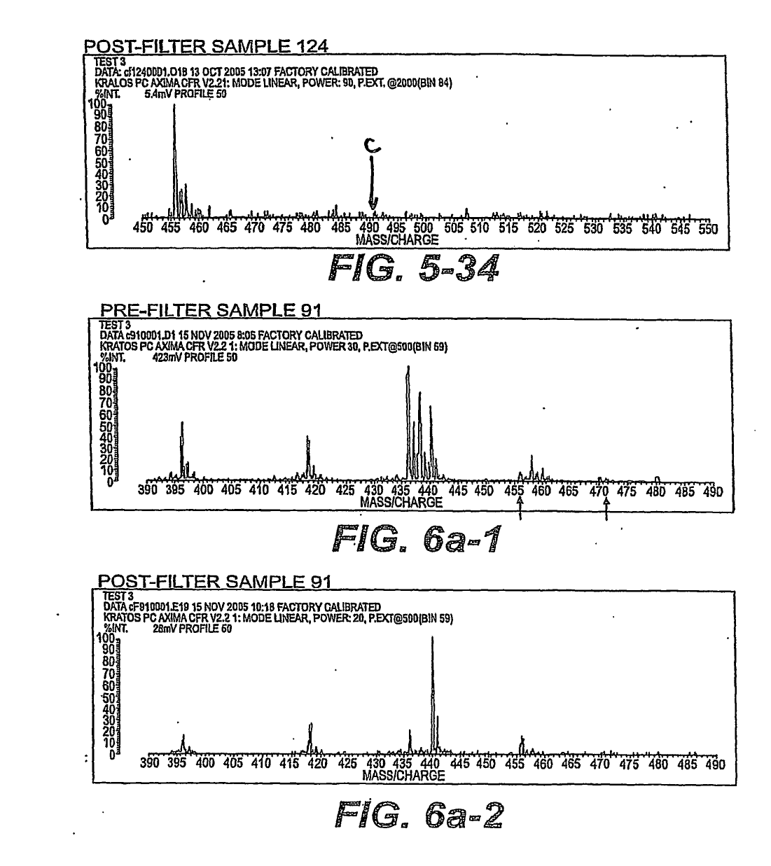

FIGS. 5-1 to 5-34 illustrate comparisons of pre-filter and post-filter samples for responders to a treatment and non-responders to a treatment. FIGS. 5-1 to 5-14 are chromatograms comparing pre-filter and post-filter samples of patients showing response to Taxol-based chemotherapy. FIGS. 5-15 to 5-34 are chromatograms comparing pre-filter and post-filter samples of patients showing no response to Taxol-based chemotherapy. Analysis of the chromatograms reveals that a peak is present at a particular point C in a substantial number of the non-responders that is not present in the responders. As shown in the figures, point C corresponds to a mass over charge ratio of 491. In the present example, the presence of a peak at point C illustrates the non-response to Taxol-based chemotherapy. Therefore, it can be predicted, using the methods of the present invention, that patients whose filtered chromatogram shows a peak at point C may be predicted to be a non-responder to Taxol-based chemotherapy.

One of ordinary skill in the art will realize that, while the above example specifically discussed Taxol-based chemotherapy, the present invention is not meant to be limited to any particular disease treatment. The present invention may be used to predict response to treatment for a variety of diseases including, but not limited to, treatment for cancers of the respiratory, gastrointestinal, renal, CNS, endocrine and blood systems or any other diseases or disease processes (e.g. necrosis, apoptosis) in which there are potential alterations in molecules contained in biological fluid (e.g. blood and blood derivatives, urine, cerebral spinal fluid, sputum, lavage). Such biological molecules may include, but are not limited to, macromolecules such as polypeptides, proteins, nucleic acids, enzymes, DNA, RNA, polynucleotides, oligonucleotides, carbohydrates, oligosaccharides, polysaccharides, fragments of biological Macromolecules (e.g. nucleic acid fragments, peptide fragments, and protein fragments), complexes of biological macromolecules (e.g. nucleic acid complexes, protein-DNA complexes, receptor-ligand complexes, enzyme-substrate, enzyme inhibitors, peptide complexes, protein complexes, carbohydrate complexes, and polysaccharide complexes), small biological molecules such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphospho-sugars, synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents; and synthetic polymers, oligomers, and copolymers. Additionally, any suitable mixture or combination of the substances mentioned above may also be included in the biological samples.

While the invention has been described with reference to certain exemplary embodiments thereof, those skilled in the art may make various modifications to the described embodiments of the invention without departing from the scope of the invention. The terms and descriptions used herein are set forth by way of illustration only and are not meant as limitations. In particular, although the present invention has been described by way of examples, a variety of compositions and methods would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

The foregoing descriptions of specific embodiments of the present invention are presented for purposes of illustration and description. They are not intended to be exhaustive or to limit the invention to the precise forms disclosed. Obviously, many modifications and variations are possible in view of the above teachings. While the embodiments were chosen and described in order to best explain the principles of the invention and its practical applications, thereby enabling others skilled in the art to best utilize the invention, various embodiments with various modifications as are suited to the particular use are also possible. The scope of the invention is to be defined only by the claims appended hereto, and by their equivalents.

What is claimed is:

1. A method for determining the probability of disease, comprising:
filtering a biological fluid through a hole array filter,
generating mass spectra data from the filtered biological fluid; and
comparing the mass spectra data with a database.

2. The method of claim 1, wherein the disease is lung cancer.

3. The method of claim 1, wherein the disease is bladder cancer.

4. The method of claim 1, wherein the disease is pancreatic cancer.

5. The method of claim 1, wherein the mass spectra data is taken by MALDI-TOF-MS.

6. The method of claim 1, wherein the biological fluid is serum.

7. The method of claim 1, wherein the biological fluid is urine.

8. The method of claim 1, wherein the biological fluid is plasma.

9. The method of claim 1, wherein the hole array filter comprises holes with a diameter of at least about 1 to 10 μm.

10. The method of claim 1, wherein the hole array filter includes a hole array layer having a thickness of at least about 6 to 10 μm.

11. The method of claim 1, wherein the hole array filter comprises:
a first area with a hole array; and
a second area for maintaining filter rigidity, the second area having a thickness greater than the thickness of the first area.

12. The method of claim 1, wherein the hole array filter comprises a first Si layer, an SiO₂ layer and a second Si layer, the first Si layer having a thickness greater than the thickness of the second Si layer.

13. A method of predicting response to disease treatment comprising:
generating a first set of mass spectra data from a first set of samples from a population that responds to a treatment of a disease A after filtration of the first set of samples through a hole array filter;
generating a second set of mass spectra data from a second set of samples from a population that does not respond to the same treatment of disease A after filtration of the second set of samples through a hole array filter; and
comparing corresponding peaks in first and second sets of mass spectra data, wherein a difference in corresponding peaks indicates that the peaks represent at least one marker indicating the likelihood of response to the treatment of disease A;
wherein upon identifying the at least one marker, using the at least one marker to predict the likelihood of response to the treatment of disease.

14. The method of claim 13, wherein the structure of the hole array filter through which the first set of samples are filtered and the structure of the hole array filter through which the second set of samples are filtered are substantially identical.

15. The method of claim 13, wherein each sample is filtered through a separate hole array filter.

16. The method of claim 13, wherein the hole array filter through which the first set of samples are filtered and the hole array filter through which the second set of samples are filtered each include holes with a diameter of at least about 1 to 10 μm.

17. The method of claim 13, wherein the hole array filter through which the first set of samples are filtered and the hole array filter through which the second set of samples are filtered each include a hole array layer having a thickness of at least about 6 to 10 μm.

18. The method of claim 13, wherein the hole array filter comprises:
a first area with a hole array; and
a second area for maintaining filter rigidity, the second area having a thickness greater than the thickness of the first area.

19. The method of claim 13, wherein the hole array filter comprises a first Si layer, an SiO₂ layer and a second Si layer, the first Si layer having a thickness greater than the thickness of the second Si layer.

20. The method of claim 13, wherein the first set of samples and the second set of samples are biological fluids.

21. The method of claim 20, wherein the biological fluids are serum, urine or plasma.

22. The method of claim 13, wherein disease A is lung cancer.

23. The method of claim 13, wherein disease A is bladder cancer.

24. The method of claim 13, wherein disease A is pancreatic cancer.

25. The method of claim 13, wherein the mass spectra data is generated by MALDI-TOF-MS.

26. The method of claim 13, wherein the hole array filter through which the first set of samples are filtered and the hole array filter through which the second set of samples are filtered are separate hole array filters.

27. A method of enhancing the identification of peaks in a mass spectrometric method comprising:
filtering a sample through a hole array filter, and
generating mass spectra data from the filtered sample.

28. The method of claim 27, wherein the hole array filter comprises holes with a diameter of at least about 1 to 10 μm.

29. The method of claim 27, wherein the hole array filter includes a hole array layer having a thickness of at least about 6 to 10 μm.

30. The method of claim 27, wherein the hole array filter comprises:
a first area with a hole array; and
a second area for maintaining filter rigidity, the second area having a thickness greater than the thickness of the first area.

31. The method of claim 27, wherein the hole array filter comprises a first Si layer, an SiO₂ layer and a second Si layer, the first Si layer having a thickness greater than the thickness of the second Si layer.

32. The method of claim 27, wherein the mass spectra data is generated by MALDI-TOF-MS.

33. The method of claim 27, wherein the sample is a biological fluid.

34. The method of claim 27, wherein the sample is serum.

35. The method of claim 27, wherein the sample is urine.

36. The method of claim 27, wherein the sample is plasma.

37. A method of increasing sensitivity and specificity in disease detection comprising:
generating a first set of mass spectra data from a first set of biological fluid samples from a population with disease A after filtration of the first set of biological fluid samples through a hole array filter;
generating a second set of mass spectra data from a second set of biological fluid samples from a population without disease A after filtration of the second set of biological fluid samples through a hole array filter, and
comparing the first and second sets of mass spectra data, wherein a difference between corresponding peaks in the first and second sets of mass spectra data indicates at least one disease A negative marker.

38. The method of claim 37, further comprising the step of using the at least one disease A negative marker to detect whether a patient has disease A.

39. The method of claim 37, wherein the mass spectra data is generated by MALDI-TOF-MS.

40. The method of claim 37, wherein the biological fluid samples are serum.

41. The method of claim 37, wherein the biological fluid samples are urine.

42. The method of claim 37, wherein the biological fluid samples are plasma.

43. The method of claim 37, wherein the structure of the hole array filter through which the first set of samples are filtered and the structure of the hole array filter through which the second set of samples are filtered are substantially identical.

44. The method of claim 37, wherein the hole array filter through which the first set of samples are filtered and the hole array filter through which the second set of samples are filtered each include holes with a diameter of at least about 1 to 10 μm.

45. The method of claim 37, wherein the hole array filter through which the first set of samples are filtered and the hole array filter through which the second set of samples are filtered each include a hole array layer having a thickness of at least about 6 to 10 μm.

46. The method of claim 37, wherein the hole array filter comprises:
a first area with a hole array; and
a second area for maintaining filter rigidity, the second area having a thickness greater than the thickness of the first area.

47. The method of claim 37, wherein the hole array filter comprises a first Si layer, an SiO₂ layer and a second Si layer, the first Si layer having a thickness greater than the thickness of the second Si layer.

48. The method of claim 37, wherein disease A is lung cancer.

49. The method of claim 37, wherein disease A is bladder cancer.

50. The method of claim 37, wherein disease A is pancreatic cancer.

51. The method of claim 37, wherein the hole array filter through which the first set of samples are filtered and the hole array filter through which the second set of samples are filtered are separate hole array filters.

52. The method of claim 37, wherein each sample is filtered through a separate hole array filter.

53. An apparatus for filtering biological fluid to predict response to disease treatment, comprising:
at least one hole array filter;
wherein:
a first set of samples from a population that respond to a treatment of a disease A is filtered through one of said at least one hole array filter;
a first set of mass spectra data is generated from the first set of samples after filtering through one of said at least one hole array filter,
a second set of samples from a population that does not respond to the same treatment of disease A is filtered through one of said at least one hole array filter;
a second set of mass spectra data is generated from the second set of samples after filtering through one of said at least one hole array filter, and
corresponding peaks in the first and second sets of mass spectra data are compared, wherein a difference in corresponding peaks indicates that the peaks represent at least one marker indicating the likelihood of response to the treatment of disease A.

54. The apparatus of claim 53, wherein said at least one hole array filter comprises at least one first hole array filter and at least one second hole array filter.

55. The apparatus of claim 54, wherein the first set of samples is filtered through the at least one first hole array filter and the second set of samples is filtered through the at least one second hole array filter.

56. The apparatus of claim 55, wherein each sample is filtered through a separate hole array filter.

57. The apparatus of claim 53, wherein upon identifying the at least one marker, the at least one marker is used to predict the likelihood of response to the treatment of disease.

58. The apparatus of claim 53, wherein each of said at least one hole array filter comprises holes with a diameter of at least about 1 to 10 μm.

59. The apparatus of claim 53, wherein each of said at least one hole array filter includes a hole array layer having a thickness of at least about 6 to 10 μm.

60. The apparatus of claim 53, wherein each of said at least one hole array filter comprises:
a first area with a hole array; and
a second area for maintaining filter rigidity, the second area having a thickness greater than the thickness of the first area.

61. The apparatus of claim 53, wherein each of said at least one hole array filter comprises a first Si layer, an SiO₂ layer and a second Si layer, the first Si layer having a thickness greater than the thickness of the second Si layer.

62. An apparatus for filtering biological fluid to detect disease by measuring mass spectra data of filtered biological fluid, comprising:
at least one hole array filter;
wherein:
a first set of biological fluid samples from a population with disease A are filtered through one of said at least one hole array filter;
a first Set of mass spectra data is generated from the first set of biological fluid samples after filtering through one of said at least one hole array filter,
a second set of biological fluid samples from a population without disease A are filtered through one of said at least one hole array filter;
a second set of mass spectra data is generated from the second set of biological fluid samples after filtering through one of said at least one hole array filter; and
the first and second sets of mass spectra data are compared, wherein a difference between corresponding peaks in the first and second sets of mass spectra data indicates at least one disease A negative marker.

63. The apparatus of claim 62, wherein said at least one hole array filter comprises a first hole array filter and a second hole array filter.

64. The apparatus of claim 63, wherein the first set of samples is filtered through the first hole array filter and the second set of samples is filtered through the second hole array filter.

65. The apparatus of claim 64, wherein each sample is filtered through a separate hole array filter.

66. The apparatus of claim 62, wherein each of said at least one hole array filter comprises holes with a diameter of at least about 1 to 10 μm.

67. The apparatus of claim 62, wherein each of said at least one hole array filter includes a hole array layer having a thickness of at least about 6 to 10 μm.

68. The apparatus of claim 62, wherein each of said at least one hole array filter comprises:
a first area with a hole array; and
a second area for maintaining filter rigidity, the second area having a thickness greater than the thickness of the first area.

69. The apparatus of claim 62, wherein each of said at least one hole array filter comprises a first Si layer, an SiO₂ layer and a second Si layer, the first Si layer having a thickness greater than the thickness of the second Si layer.

70. An apparatus for filtering biological fluid to enhance the identification of peaks in a mass spectrometric method, comprising:
a hole array filter,
wherein:
a biological fluid sample is filtered through said hole array filter; and
mass spectra data is generated from the filtered biological fluid sample.

71. The apparatus of claim 70, wherein said hole array filter comprises holes with a diameter of at least about 1 to 10 μm.

72. The apparatus of claim 70, wherein said hole array filter includes a hole array layer having a thickness of at least about 6 to 10 μm.

73. The apparatus of claim 70, wherein said hole array filter comprises:
a first area with a hole array; and
a second area for maintaining filter rigidity, the second area having a thickness greater than the thickness of the first area.

74. The apparatus of claim 70, wherein each of said at least one hole array filter comprises a first Si layer, an SiO₂ layer and a second Si layer, the first Si layer having a thickness greater than the thickness of the second Si layer.
